(19) United States
(12) Patent Application Publication
(10) Pub. No.: US 2012/0045514 A1
(43) Pub. Date: Feb. 23, 2012

(54) ANTI-CANCER MICROPARTICLE

(75) Inventors: Yin Win Khin, Singapore (SG); Enyi Ye, Singapore (SG); Mingyong Han, Singapore (SG)

(73) Assignee: Agency for Science, Technology and Research, Singapore (SG)

(21) Appl. No.: 13/130,957

(22) PCT Filed: Nov. 24, 2009
(86) PCT No.: PCT/SG2009/000447

§ 371 Date: Oct. 18, 2011

Related U.S. Application Data
(60) Provisional application No. 61/117,292, filed on Nov. 24, 2008.

(51) Int. Cl. A61K 9/14; C12Q 1/02; A61P 35/00; C12N 5/09; B82Y 15/00; B82Y 5/00

(52) U.S. Cl. 424/489; 424/400; 435/375; 435/29; 977/774; 977/915; 977/927

(57) ABSTRACT

The present invention relates to a microparticle and its use. The microparticle has a width of at least about 1 micron. The microparticle includes a biocompatible polymer, a nanoparticle, and an anti-cancer agent.

### CROSS REFERENCE TO RELATED APPLICATIONS

This application makes reference to and claims the benefit of priority of an application for “Size Dependent Cellular Uptake And Targeting Effect Of Microparticulate Carriers In Cancer Diagnostics And Chemotherapy” filed on Nov. 24, 2008 with the United States Patent and Trademark Office, and there duly assigned U.S. Provisional Ser. No.61/117,292. The contents of said application filed on Nov. 24, 2008 is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates to an anti-cancer micoparticle. The microparticle includes an anti-cancer agent and a nanocrystal.

### BACKGROUND OF THE INVENTION

Cancer is a major cause of death worldwide, being the second-leading cause of death in developed countries and even the number one cause of death in e.g. Australia, Japan, Korea, Singapore and the male population of the UK and Spain. The number of people who develop cancer each year is increasing. No substantial improvement in cancer treatment has been achieved over the last 50 years, partly because of the limitations in the existing drugs, as well as absence of an efficient drug delivery system. Since development of new anticancer drugs is very expensive and time-consuming, emergence of new methods/devices for enhanced delivery of current drugs to the desired disease site is crucial. Simple and efficient diagnostic tools are also in high demand for early detection and monitoring of treatment progress.

Controlled release technologies have received more and more attention as they provide many advantages, such as prolonged efficacy time, less side effects, and improved bioavailability, in comparison with conventional dosage forms. In recent years, particulate drug delivery systems have emerged as one of the most promising approaches to achieve site-specific or targeting drug delivery, and been a major focus in the pharmaceutical industry. The achievement of controlled release of pharmaceutically active agents to the specific site of action at the therapeutically optimal rate and dose regimen has been a major goal in designing such delivery systems. Various polymers have been used as carrier materials in drug delivery research, of which biocompatible and biodegradable polymers, such as FDA approved poly(DL-lactide-co-glycolide) (PLGA), are commonly used.

Nanoparticles have been the focus of recent research efforts, since many cell types are known to be capable of ingesting small-sized matter of lower than about 200 nm by mechanisms such as pinocytosis and receptor- and chlathrin-mediaetd endocytosis. Low molecular weight compounds, peptides, proteins and nucleic acids can be loaded into nanoparticles that are not recognized by the immune system. Polymer coated quantum dots have been used to target cancer cells by means of an antibody immobilized thereon (Gao, X., et al., Nature Biotech. (2004) 22, 969-976). The quantum dots' properties could further be used to trace the quantum dots in the body. The use of nanocrystals often bears a health risk due to e.g. cytotoxic side effects or hemolysis. To address cytotoxic side effects of nanocrystal use and to improve imaging properties optimization efforts regarding the polymer used for coating have been carried out (Pan, J., et al., Biotechnology & Bioengineering (2008) 101, 622-632). Identifying suitable targeting-ligands for cancer cells that have little or no side effects and immobilizing such ligands further requires tedious efforts in terms of cost and time.

Accordingly, it is an object of the present invention to provide means and a method of delivering an anti-cancer agent to cancer cells in the body that overcomes at least some of the above mentioned difficulties and draw-backs. Ideally such means and method also provides the option of tracing the delivery of the anti-cancer agent, or the means used in this regard, in the body. This object is solved by providing a microparticle as defined in claim 1.

### SUMMARY OF THE INVENTION

In a first aspect the present invention provides a microparticle. The microparticle has a width of at least about 1 micron. The microparticle includes a biocompatible polymer and a nanoparticle. Further, the microparticle includes an anti-cancer agent or a pharmaceutically acceptable salt, ester, prodrug or hydrate thereof.

In a second aspect the present invention relates to the use of a microparticle according to the first aspect in the manufacture of a medicament for treating cancer.

In a third aspect the present invention provides a process of forming a microparticle. The microparticle has a width of at least about 1 micron. The microparticle includes a nanoparticle. The process includes providing a polar solvent. The process also includes providing a non-polar solvent. The polar solvent and the non-polar solvent are at least substantially immiscible with each other. The process further includes providing a biocompatible polymer. Furthermore, the process includes dissolving the biocompatible polymer in one of the polar solvent and the non-polar solvent. The process also includes providing a nanoparticle. Further, the process includes dispersing the nanoparticle in one of the polar solvent and the non-polar solvent. Furthermore, the process includes combining the non-polar solvent and the polar solvent. Thereby a two-phase mixture is formed. The process includes agitating the two-phase mixture, for example by means of static agitation or mechanical agitation. The process thereby includes allowing the formation of a microparticle.

In a fourth aspect the present invention relates to a method of treating cancer. The method includes administering to an organism suffering from cancer a microparticle. The microparticle includes a biocompatible polymer and an anti-cancer agent or a pharmaceutically acceptable salt, ester, prodrug or hydrate thereof. Thereby typically a therapeutically effective amount of the anti-cancer agent, pharmaceutically acceptable salt, ester, prodrug or hydrate is administered. The microparticle has a width of at least about 1 micron. In some embodiments the microparticle is a microparticle according to the first aspect.

In a related aspect the invention provides a method of selectively directing an anti-cancer agent, or a pharmaceutically acceptable salt, ester, prodrug or hydrate thereof, to one or more cancer cells within a plurality of healthy cells. The method includes providing a cell population. The cell population includes a plurality of healthy cells and one or more cancer cells. The method also includes contacting the cell population with a microparticle. The microparticle includes a biocompatible polymer and an anti-cancer agent or a pharmaceutically acceptable salt, ester, prodrug or hydrate thereof. The microparticle has a width of at least about 1 micron.

### BRIEF DESCRIPTION OF THE DRAWINGS

The invention will be better understood with reference to the detailed description when considered in conjunction with non-limiting examples and the accompanying drawings, in which:

FIG. 1 depicts the use of drug carrier particles with antibodies immobilized thereon in the art. The antibody directs the particle from extracellular fluid (a) to target cells (b).

FIG. 2 depicts the current use of quantum dots that are internalized into a cell.

FIG. 3 is a schematic depicting the steps of endocytosis of quantum dots (1) that allows crossing the plasma membrane (2).

FIG. 4A: Particles of QD size are taken up by both healthy cells (3) and cancer cells (4). FIG. 4B: Microparticles according to the invention are mostly taken up by cancer cells (4) compared to healthy tells (3).

FIG. 5 depicts the formation of microparticles of the invention. QDs can be incorporated into PLGA nanoparticles (5) and PLGA microparticles (6) in a coating process.

FIG. 6A shows a SEM image of the PLGA nanoparticles on a glass substrate.

FIG. 6B is a fluorescent photograph of the PLGA nanoparticles in aqueous solution under UV excitation.

FIG. 6C is a confocal image depicting internalization of green PLGA nanoparticles into the cytoplasm of CCD-112CoN cells after 2-h incubation at 37° C. followed by counterstaining of nucleus with red propidium iodide (only green channel shown).

FIG. 6D is the image of FIG. 6C with all channels shown, i.e. nucleus and nanoparticles.

FIG. 7A depicts the cellular uptake of green QD-loaded PLGA nanoparticles into human non-small lung cancer cells, NCI-H1299, with counterstaining of nucleus with red propidium iodide (only green channel shown).

FIG. 7B is the image of FIG. 7A with all channels shown, i.e. nucleus and nanoparticles.

FIG. 8A depicts the cellular uptake of green QD-loaded PLGA nanoparticles into human breast cancer cells, MCF-7, with counterstaining of nucleus with red propidium iodide (only green channel shown).

FIG. 8B is the image of FIG. 8A with all channels shown, i.e. nucleus and nanoparticles.

FIG. 9A shows the cellular uptake of red QD-loaded PLGA nanoparticles into rat glioma cells, C6, with counterstaining of nucleus with blue DAPI (only red channel shown).

FIG. 9B is the image of FIG. 9A with all channels shown, i.e. nucleus and nanoparticles.

FIG. 10A shows the cellular uptake of green QD-loaded PLGA nanoparticles into human normal colon cells, CCD-112CoN, with counterstaining of nucleus with red PI (only green channel shown).

FIG. 10B is the image of FIG. 10A with all channels shown, i.e. nucleus and nanoparticles.

FIG. 11A shows the cellular uptake of cyan-colored QD-loaded PLGA nanoparticles into human normal colon cells, CCD-112CoN, with counterstaining of nucleus with blue DAPI (only cyan channel shown).

FIG. 11B is the image of FIG. 11A with all channels shown, i.e. nucleus and nanoparticles.

FIG. 12 shows an SEM image of nearly uniform microparticles, which include hydrophobic QDs, after size separation.

FIG. 13A shows a confocal image of CCD-112CoN, non-cancer cells, after 2-h incubation at 37° C. with green hydrophobic QDs, followed by counterstaining of the nucleus with red PI. Only the red channel is shown.

FIG. 13B is the image of FIG. 13A with only the green channel shown.

FIG. 13C is the image of FIG. 13A with all channels shown.

FIG. 14A shows a confocal image of breast cancer cells, MCF-7, after 2-h incubation at 37° C. with green hydrophobic QDs, followed by counterstaining of nucleus by red PI. Only the red channel is shown.

FIG. 14B is the image of FIG. 14A with only the green channel shown.

FIG. 14C is the image of FIG. 14A with all channels shown.

FIG. 15 shows intracellular distribution of microparticles by a series of confocal images of MCF-7 cell sectioned at different XY planes with 1 μm interval steps along the Z direction, after cells were incubated with green QD-incorporated PLGA microparticles, cell nuclei are counterstained red with PI.

FIG. 16A is a confocal image of CCD-112CoN cells after 4-h incubation with DOX-nanoparticles at 37° C. The inset represents fluorescence intensity profiles of DOX (upper curve) and DAPI (lower curve) in the counter-stained CCD-112CoN cells with DAPI.

FIG. 16B shows the cumulative release of DOX from the DOX-nanoparticles in pH 7.4 PBS buffer solution at 37° C.

FIG. 16C depicts a confocal image of CCD-112CoN cells after 4-h incubation with DOX in the amounts after 4-h release from the DOX-nanoparticles.

FIG. 16D depicts a confocal image of CCD-112CoN cells after 4-h incubation with DOX in the amounts after complete release from the DOX-nanoparticles.

FIG. 17A is a confocal image for cross-section (in XY plane, top left image) of a green QD-incorporated PLGA microparticle of the invention in the XZ (bottom left image) and YZ (top right image) plane.

FIG. 17B is a schematic illustration of a QD- and drug-loaded microparticle, indicating the locations of QD and drug inside the microparticle.

FIG. 17C is a confocal image of an MCF-7 cell after incubation with blue drug- and green QD-incorporated PLGA microparticles, and staining of cell nuclei with red PI (all channels shown).

FIG. 17D depicts the image of FIG. 17C with only the green channel shown.

FIG. 17E depicts the image of FIG. 17C with only the blue channel shown.

FIG. 17F depicts the image of FIG. 17C with both green and blue channels (but not the red channel).

FIG. 18A depicts a confocal image of fluorescent drug incorporated microparticles.

FIG. 18B depicts a confocal image of a MCF-7 cell after incubation with blue drug-incorporated microparticles, the cell nucleus is counterstained red with PI (all channels shown).

FIG. 18C is the confocal image of FIG. 18B with only the blue channel shown.

FIG. 18D depicts cell viability after being treated up to 4 days with control placebo particles (blank), QD-loaded microparticles (QD), and drug-loaded microparticles (CPT).

FIG. 19A is a confocal image of CCD-112CoN cells (non-cancer cells) after treatment with fluorescent drug-loaded microparticles and counterstaining cell nuclei with red PI (blue and red channel images shown).

FIG. 19B is the confocal image of FIG. 19A with only the red channel shown.

FIG. 19C is the confocal image of FIG. 19A with only the blue channel shown.

FIG. 20A is a confocal image of NIH3T3 cells (non-cancer cells) after treatment with fluorescent drug-loaded microparticles and counterstaining cell nuclei with red PI (blue and red channel images shown).

FIG. 20B is the confocal image of FIG. 20A with only the blue channel shown.

FIG. 20C is the confocal image of FIG. 20A with only the red channel shown.

FIG. 21A is a confocal image of C6 cells (cancer cells) after treatment with fluorescent drug-loaded microparticles and counterstaining cell nuclei with red PI (blue and red channel images shown).

FIG. 21B is the confocal image of FIG. 21A with only the blue channel shown.

FIG. 21C is the confocal image of FIG. 21A with only the red channel shown.

### DETAILED DESCRIPTION OF THE INVENTION

The present invention provides a microparticle. As used herein, the term “micro-particle” refers to a microscopic particle with a size measured in micrometres (μm). Typically, the microparticle has an average width, including diameter, of from about 1 μm to about 100 μm, such as an average width of from about 1 μm to about 50 μm, for instance from about 1 μm to about 25 μm. In some embodiments the microparticle has a width of maximally about 5 microns. In some embodiments the microparticle has a width of a width of at least about 2 microns. In particular, the microparticle may have an average width of from about 1 μm to about 10 μm, such as about 1 μm to about 7 μm, about 1 μm to about 6 μm, about 1 μm to about 5 μm, about 1.5 μm to about 5 μm, about 2 μm to about 5 μm, about 2 μm to about 6 μm, about 2.5 μm to about 5 μm, about 2.5 μm to about 6 μm, about 3 μm to about 6 μm, about 3 μm to about 5 μm, about 4 μm to about 6 μm or about 4 μm to about 5 μm.

The microparticle may in some embodiments be a member of a plurality of microparticles. Such a plurality of microparticles may be of, or at least essentially of, a selected width range. As a few illustrative examples, the microparticles may have, e.g. at least essentially, have a width of a range of about 2 to about 5 μm, about 3 to about 5 μm, of about 3.5 to about 5 μm, or about 4 to 5 μm. A respective plurality of microparticles may in some embodiments also be of, or at least essentially of, the same width. As a few illustrative examples, all or at least essentially all microparticles may be of a width of about 2 μm, of about 2.5 μm, of about 3 μm, of about 3.5 μm, of about 4 μm, of about 4.5 μm or of about 5 μm. Any technique available in the art can be employed to obtain microparticles of a desired width or of a desired width range, such as for example size-selective fractionation.

The microparticle is of solid or semi-solid matter that allows including the anti-cancer agent into the microparticle. The microparticle may in some embodiments be a microsphere, i.e. a matrix-filled system without a void or cavity. In some embodiments the microparticle may be of non-homogenous structure. As an illustrative example, the microparticle may have a core loaded with the anti-cancer agent, wherein the core has a different composition of matter, e.g. of biocompatible polymer, than a shell of the microparticle. Such a shell may for instance be formed after the formation of the core, for example using a spray dryer. In some embodiments a respective shell may include the anti-cancer agent. The microparticle includes a biocompatible polymer. The term “biocompatible polymer” (which also can be referred to as “tissue compatible polymer”), as used herein, is a polymer that produces little if any adverse biological response when used in vivo. The term thus generally refers to the inability of a polymer to promote a measurably adverse biological response in a cell, including in the body of an animal, including a human. A biocompatible polymer can have one or more of the following properties: non-toxic, non-mutagenic, non-allergenic, non-carcinogenic, and/or non-irritating. A biocompatible polymer, in the least, can be innocuous and tolerated by the respective cell and/or body. A biocompatible polymer, by itself, may also improve one or more functions in the body. A variety of biocompatible polymers is suitable for the formation of a microparticle according to the invention. The biocompatible polymers can be synthetic polymers, naturally occurring polymers or combinations thereof. As used herein the term “synthetic polymer” refers to polymers that are not found in nature, including polymers that are made from naturally occurring biomaterials. Examples of suitable biocompatible polymers include non-absorbable polymers such as polypropylene, polyethylene, polyethylene terephthalate, polytetrafluoroethylene, polyaryl-etherketone, nylon, fluorinated ethylene propylene, polybutester, and silicone, or copolymers thereof (e.g., a copolymer of polypropylene and polyethylene); absorbable polymers such as polyglycolic acid (PGA), polylactic acid (PLA), polycaprolactone, and polyhydroxy-alkanoate, copolymers thereof (e.g., a copolymer of PGA and PLA), and mixtures thereof.

A wide variety of biodegradable polymers is also suitable for a microparticle of the invention. Biodegradable polymers, as defined herein, are a subset of biocompatible polymers that gradually disintegrate or are absorbed in vivo over a period of time (e.g., within months or years). Disintegration may for instance occur via hydrolysis, may be catalysed by an enzyme and may be assisted by conditions to which the microparticles are exposed in the cell. Examples of biodegradable polymers suitable for a microparticle according to the invention include, but are not limited to, polyesters, polyanhydrides, polyorthoesters, polyphosphazenes, polyphosphates, polyphosphoesters, polyphosphonates, polydioxanones, polyhydroxyalkanoates, polycarbonates, polyalkylcarbonates, polyorthocarbonates, polyesteramides, polyamides, polyamines, polypeptides, polyurethanes, polyetheresters, or combinations thereof. An illustrative example of a biodegradable polymer is poly-(α-hydroxy acid), for example polylactic acid, polyglycolic acid and copolymers and mixtures thereof such as poly(L-lactide) (PLLA), poly(D,L-lactide) (PLA); poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), poly(D,L-lactide-co-caprolactone) (PLA/PCL), poly(glycolide-co-capro-lactone) (PGA/PCL); polyethylene oxide (PEO); polydioxanone (PDS); polypropylene fumarate; poly(ethyl glutamate-co-glutamic acid); poly(tert-butyloxy-carbonylmethyl glutamate); poly(carbonate-esters). Further examples of suitable biodegradable polymers include a polylacton such as a poly(c-caprolactone) (PCL) and copolymers thereof such as polycaprolactone co-butylacrylate; polyhydroxybutyrate (PHBT) and copolymers of polyhydroxybutyrate; poly(phosphazene); poly(phosphate ester); a polypeptide; a polydepsipeptide, a maleic anhydride copolymer; a poly-phosphazene; a polyiminocarbonate; poly(dimethyl-trimethylene carbonate-co-trimethylene carbonate); a polydioxanone, polyvalerolactone, a polyorthoester, a polyanhydride, polycyanoacrylate; a tyrosine-derived polycarbonate or polyester-amide; a polysaccharide such as hyaluronic acid; and copolymers and mixtures of the above polymers, among others. In some embodiments the biocompatible polymer may be crosslinked, for example to improve mechanical stability of the microparticle.

In certain embodiments, the microparticle is formed from a biocompatible polymer, such as a biodegradable polymer. It may for instance be formed from a poly(a-hydroxy acid), such as a poly(lactide) (“PLA”), a copolymer of lactide and glycolide, such as a poly(D,L-lactide-co-glycolide) (“PLG”), or a copolymer of D,L-lactide and caprolactone. Poly(D,L-lactide-co-glycolide) polymers include those having a lactide/glycolide molar ratio ranging, for example, from 20:80 to 25:75 to 40:60 to 45:55 to 55:45 to 60:40 to 75:25 to 80:20, and having a molecular weight ranging, for example, from 5,000 to 10,000 to 20,000 to 40,000 to 50,000 to 70,000 to 100,000 to 200,000 Daltons. The microparticle may also include or be formed from poly (lactic acid)-d-α-tocopheryl polyethylene glycol 1000 succinate (e.g. Pan, J., et al., Biotechnology & Bioengineering (2008) 101, 622-633). Yet further illustrative examples of a biocompatible polymer are a collagen, a chitosan, an alginate, heparin, gelatine and hyaluronic acid, six naturally occurring polymers. In this regard polyhydroxybutyrate (supra) is a polyester produced as granules by microorganisms.

The microparticle further includes a nanoparticle. The nanoparticle may for example be a nanocrystal or an amorphous nanoparticle. The nanoparticle, e.g nanocrystal, is generally of inorganic matter. In some embodiments the nanoparticle includes paramagnetic matter. In some embodiments the nanoparticle includes ferromagnetic matter (also termed superparamagnetic). Examples of ferromagnetic matter include, but are not limited to iron, nickel, and cobalt. In some embodiments the nanoparticle includes matter that can have ferromagnetic properties under certain conditions. In some embodiments the nanoparticle is at least essentially free of ferromagnetic paramagnetic matter, i.e. matter that is ferromagnetic at standard conditions (atmospheric pressure, temperature about 18° C.) including entirely free thereof. In some embodiments the nanoparticle is at least essentially free of paramagnetic matter, including entirely free thereof. Examples of paramagnetic matter include, but are not limited to magnesium, molybdenum, lithium, and tantalum. In some embodiments the nanoparticle includes diamagnetic matter.

In some embodiments the inorganic matter is or includes a semiconductor. Such a nanoparticle is typically a quantum dot (QD). Respective nanoparticles that confine the motion of conduction band electrons, valence band holes, or excitons (in all three spatial directions) can serve as “droplets” of electric charge and are termed quantum dots. Quantum dots can be as small as 2 to 10 nanometers, with self-assembled quantum dots typically ranging between 10 and 50 nanometers in size. Quantum dots in the microparticles of the invention may be of any desired size, e.g. selected in the range from about 1 to about 200 nanometers, from about 1 to about 150 nanometers, from about 2 to about 150 nanometers, from about 1 to about 100 nanometers, from about 2 to about 100 nanometers, from about 1 to about 80 nanometers or from about from about 2 to about 80 nanometers. Quantum dots have attracted interest for various uses, including electronics, fluorescence imaging and optical coding. They are of particular importance for optical applications due to their theoretically high quantum yield. A respective QD may be of any desired shape, including ball-shaped, i.e. spherical, or rod shape.

In accordance with the invention, any suitable type of nanoparticle (quantum dot) can be rendered water soluble, so as long as the surface of the nanoparticle can interact, for example, via hydrophobic interactions or van-der-Waals interactions, with the biocompatible polymer, which may be an amphiphilic polymer.

A well established route that can be used to prepare high-quality semiconductor quantum dots is the decomposition of molecular precursors at high temperatures in a coordinating solvent (for an overview of previous techniques see e.g. Reed, M. A., Scientific American (1993), January, 118-123), possibly, in the presence of a negative ion source, e.g., TOP/Se, TOP/S, etc. This process was developed in 1993 by Murray et al. (J. Am. Chem. Soc. (1993), 115, 8706-8715) and yields quantum dots of CdE (E=Se, S, and Te). It involves the formation of a solution of dimethylcadmium in tri-n-octylphosphine (TOP) and a solution of the corresponding chalcogenide in TOP. The solutions are combined and rapidly injected into tri-n-octylphosphine oxide (TOPO) at high temperatures (˜200° C.-300° C.). Thereby TOP/TOPO capped nano-particles are obtained. The capping agent allows particle solubility in organic solvents, and plays a crucial role in preventing particle aggregation and electronically passivating the semiconductor surface. This so-called TOPO method permits the production of highly monodisperse nanoparticles in quantities of hundreds of milligrams in one single experiment.

In one embodiment, suitable nanoparticles are nanocrystals with a nanocrystal core that includes a metal (M1) alone. For this purpose, M1 may be selected from the group consisting of an element of main group II, subgroup VIIA, subgroup VIIIA, subgroup IB, subgroup IIB, main group III or main group IV of the periodic system of the elements (PSE). Accordingly, the nanocrystal core may consist of only the metal element M1; the non-metal element A or B, as defined below, is absent. In this embodiment, the nanocrystal consists only of a pure metal from any of the above groups of the PSE, such as gold, silver, copper (subgroup Ib), titanium (subgroup IVb), terbium (subgroup Mb), cobalt, platinum, rhodium, ruthenium (subgroup VIIIb), lead (main group IV) or an alloy thereof.

In another embodiment, the nanocrystal core used in the present invention may include two elements. Accordingly, the nanocrystal core may be a binary nanocrystal alloy that includes two metal elements, M1 and M2, such as any well-known core-shell nanocrystal formed from metals such as Zn, Cd, Hg, Mg, Mn, Ga, In, Al, Fe, Co, Ni, Cu, Ag, Au and Au.

Another type of binary nanocrystals suitable in the present invention may include one metal element M1, and at least one element A selected from main group V or main group VI of the PSE. Accordingly, the one type of nanocrystal suitable for use presently has the formula M1A. Examples of such nanocrystals may be group II-VI semiconductor nanocrystals (i.e. nanocrystals including a metal from main group II or subgroup IIB, and an element from main group VI) wherein the core and/or the shell includes CdS, CdSe, CdTe, MgTe, ZnS, ZnSe, ZnTe, HgS, HgSe, or HgTe. The nanocrystal core may also be any group III-V semiconductor nanocrystal (i.e. nanocrystals including a metal from main group III and an element from main group V). Each of both of the core and/or the shell may, for example, include GaN, GaP, GaAs, GaSb, InN, InP, InAs, InSb, AlN, AlP, AlAs, AlSb. Specific examples of core shell nanocrystals that can be used in the present invention include, but are not limited to, (CdSe)-nanocrystals having a ZnS shell, as well as (CdS)-nanocrystals having ZnS shell.

The invention is not limited to the use of the above-described core-shell nanocrystals. In another embodiment, the nanocrystal of the invention can have a core consisting of a homogeneous ternary alloy having the composition M1₁₋ₓM2ₓA, wherein

(a) M1 and M2 are independently selected from an element of subgroup IIb, subgroup VIIa, subgroup VIIIa, subgroup Ib or main group II of the periodic system of the elements (PSE), when A represents an element of the main group VI of the PSE, or
(b) M1 and M2 are both selected from an element of the main group (III) of the PSE, when A represents an element of the main group (V) of the PSE.

In another embodiment nanocrystals consisting of a homogeneous quaternary alloy can be used. Quaternary alloys of this type have the composition M1₁₋ₓM2ₓA_{y}B_{1-y}, wherein

(a) M1 and M2 are independently selected from an element of subgroup IIb, subgroup VIIa, subgroup VIIIa, subgroup Ib or main group II of the periodic system of the elements (PSE), when A and B both represent an element of the main group VI of the PSE, or

(b) M1 and M2 are independently selected from an element of the main group (III) of the PSE, when A and B both represent an element of the main group (V) of the PSE.

Examples of this type of homogenous ternary or quaternary nanocrystals have been described, for instance, in Zhong et al, J. Am. Chem. Soc (2003) 125, 8598-8594, Zhong et al, J. Chem. Soc (2003) 125, 13559-13553, or the International patent application WO 2004/054923.

Such ternary nanocrystals are obtainable by a process that includes forming a binary nanocrystal M1A by

i. heating a reaction mixture containing the element M1 in a form suitable for the generation of a nanocrystal to a suitable temperature T1, adding at this temperature the element A in a form suitable for the generation of a nanocrystal, heating the reaction mixture for a sufficient period of time at a temperature suitable for forming said binary nanocrystal M1A and then allowing the reaction mixture to cool, and

ii. reheating the reaction mixture, without precipitating or isolating the formed binary nanocrystal M1A, to a suitable temperature T2, adding to the reaction mixture at this temperature a sufficient quantity of the element M2 in a form suitable for the generation of a nanocrystal, then heating the reaction mixture for a sufficient period of time at a temperature suitable for forming said ternary nanocrystal M1₁₋ₓM2ₓA and then allowing the reaction mixture to cool to room temperature, and isolating the ternary nanocrystal M1₁₋ₓM2ₓA.

In these ternary nanocrystals, the index x may have a value of 0.001<x<0.999, for example of 0.01<x<0.99, 0.1<0.9 or of 0.5<x<0.95. In other embodiments, x can have a value between about 0.2 or about 0.3 to about 0.8 or about 0.9. In quaternary nanocrystals, y may have a value of 0.001<y<0.999, for example of 0.01<y<0.99, or of 0.1<x<0.95 or between about 0.2 and about 0.8.

In such II-VI ternary nanocrystals, the elements M1 and M2 included therein may be independently selected from the group consisting of Zn, Cd and Hg. The element A of the group VI of the PSE in these ternary alloys is preferably selected from the group consisting of S, Se and Te. Thus, all combinations of these elements M1, M2 and A are within the scope of the invention. In illustrative embodiments nanocrystals used in the present invention have the composition ZnₓCd₁₋ₓSe, ZnₓCd₁₋ₓS, ZnₓCd₁₋ₓTe, HgₓCd₁₋ₓSe, HgₓCd₁₋ₓTe, HgₓCd₁₋ₓS, ZnₓHg₁₋ₓSe, ZnₓHg₁₋ₓTe, and ZnₓHg₁₋ₓS.

In some illustrative embodiments, x as used in the above chemical formulas has a value of 0.10<x<0.90 or 0.15<x<0.85, and more preferably a value of 0.2<x<0.8. In particularly preferred embodiments, the nanocrystals have the composition ZnₓCd₁₋ₓS and ZnₓCd₁₋ₓSe. Such nanocrystals are preferred in which x has a value of 0.10<x<0.95, and more preferably a value of 0.2<x<0.8.

In certain embodiments in which the nanocrystal core is made from III-V nanocrystals of the invention, each of the elements M1 and M2 are independently selected from Ga and In. The element A may be selected from P, As and Sb. All possible combinations of these elements M1, M2 and A are within the scope of the invention. In some illustrative embodiments, nanocrystals have the composition GaₓIn₁₋ₓP, GaₓIn₁₋ₓAs and GaₓIn₁₋ₓAs.

In one embodiment the nanoparticle, e.g. nanocrystal, includes semiconducting material, wherein as explained above the semiconducting material may include at least one of a metal and a metalloid.

In one embodiment, the nanoparticle is a coordination nanoparticle that may for instance have a coordination nanonetwork of nucleotides and lanthanide ions and include a dye molecule. An example of such a nanoparticle that has a fluorescence dye has been disclosed by Nishiyabu, R., et al., Angew. Chem. Int. Ed. (2009) 48, DOI: 10.1002/anie. 200904124).

The nanoparticle can serve in detecting, imaging and monitoring the delivery of the microparticles, for instance as contrast agents. As an illustrative example, in a tissue sample or in cell culture live cell imaging can be performed, thereby tracking and monitoring the whereabouts of microparticles and their amount in the cells under investigation. In this regard, photoacoustic (PA) and photothermal (PT) microscopy may be employed—which are not sensitive to scattering and autofluorescent backgrounds, since most light energy absorbed by QDs is eventually converted into heat. Using a pulse laser and a multimodule PT-PA-fluorescent microscope, Shashkov et al. (Nano Lett. (2008) 8, 3953-3958) have demonstrated this technique.

In some embodiments the microparticle of the invention includes a plurality of nanoparticles, e.g. nanocrystals. The nanoparticles may be included in the microparticle in an amount of about 0.01 to about 60 wt % of the biocompatible polymer, such as about 0.1 to about 60 wt %, about 0.1 to about 50 wt %, about 0.1 to about 45 wt %, about 0.1 to about 40 wt %, about 0.1 to about 35 wt %, about 0.1 to about 30 wt %, about 0.1 to about 25 wt %, about 0.1 to about 20 wt %, about 1 to about 20 wt %, about 2 to about 20 wt %, about 5 to about 20 wt or about 10 to about 20 wt % of the biocompatible polymer.

Preparation of high-quality quantum dots is in many cases performed at elevated temperatures in the presence of tri-n-octyl phosphine oxide (TOPO) as the stabilizing ligand. As a result the nanoparticles are coated with a monolayer of TOPO, a hydrophobic molecule, and the QDs are solvable in non-polar solvents and do not disperse in aqueous solutions.

In some embodiments the nanoparticles are distributed non-uniformly in the microparticle. In some embodiments all or at least essentially all nanoparticles of the plurality of nanoparticles are arranged close to the microparticle surface or proximate the surface of the microparticle. In some embodiments all, at least essentially all, or most of the nanoparticles are in contact with the surface of the microparticle. All, at least essentially all, or most of the nanoparticles may in some embodiments be arranged in a near-surface zone of the microparticle. This near-surface zone is defined by the distance of 0 to about 250 nm from the surface of the microparticle, i.e. the portion immediately below the surface of the microparticle.

The distribution of the nanoparticles within the microparticle of the invention can be controlled by the stirring speed upon their formation and the amount of nanoparticles used. Where the microparticles are formed by a process that involves emulsification with a non-polar solvent and an aqueous solution the volume ratio of non-polar phase and aqueous phase can also be used to control the distribution of nanoparticles within the microparticle. Where the nanoparticles are non-polar, which is typically the case for nanocrystals, they are of higher affinity to non-polar solvents than to polar solvents. This effect can be employed to obtain microparticles with nanoparticles that are non uniformly distributed therein. Nanoparticles will be more densely distributed near and at the outer part, i.e. proximate the surface, of the microparticle—rather than positioned at the inner core of the microparticle matrix—the higher the volume ratio of non-polar to polar phase. An increase of agitation, e.g. stirring speed, also causes the position of a nanoparticle within the microparticle of the invention to be closer to its periphery, i.e. closer to the surface of the microparticle. Further, where a plurality of nanoparticles is used and a microparticle with more than one nanoparticle is obtained, the amount of nanoparticles used affects their distribution in the obtained microparticle(s). The less nanoparticles are used the more will the position of a nanoparticles within the microparticle be in proximity to the surface of the microparticle. The desired location and distribution of the nanoparticles in a microparticle of the invention can be determined empirically by varying these parameters and is within the ability of the person of average skill in the art.

Where the nanoparticles are mostly situated in the periphery and near the surface of the microparticle(s), identification of each particle is rendered easy by the corresponding fluorescent ring-like morphology in the bio-imaging analyses. Further, this structure of the microparticle(s) enables quantitative analyses which cannot be achieved otherwise. These ‘smart microparticles’ can for instance be prepared by a modified emulsification/solvent-evaporation method and formulated by using the biocompatible amphiphilic copolymer, poly (DL-lactide-co-glycolide) or PLGA, as the matrix material, hydrophobic QDs or other nanoparticles such as magnetic or metal ones, as the morphology-visible surface-fluorescent or functional material, and/or anticancer drugs, hydrophilic doxorubicin and hydrophobic camptothecin, as the controlled release therapeutic agent.

As regards the anti-cancer agent that is included in the microparticle of the present invention, the anti-cancer agent is typically present in an amount of at least about 0.5 weight %, such as about 1.0 weight % based on the total weight of the polymer of the microparticle. In some embodiments the anti-cancer agent is present in an amount of at least 1 about 2 weight %, for instance at least about 1.3 weight %, based on the total weight of the polymer of the microparticle. In some embodiments, the anti-cancer agent is present in an amount of at least about 2.0 weight % based on the total weight of the polymer of the microparticle, such as in an amount of at least about 3.0 weight %. The anti-cancer agent is in some embodiments present in an amount of at most about 25 weight %, such as at most about 17 weight %, at most about 15 weight %, at most about 12 weight %, at most about 10 weight %, or at most about 8 weight %, based on the total weight of the polymer of the microparticle.

The anti-cancer agent may be any desired compound or combination of compounds. In some embodiments the anti-cancer agent is or includes a low molecular weight organic compound. In some embodiments the anti-cancer agent is or includes a peptide, a protein, a lipid, a saccharide or a polysaccharide. The anti-cancer agent may be more or less homogenously distributed, e.g. dispersed, within the microparticle. In some embodiments the anti-cancer agent is located within a certain portion of the microparticle, such as a core or a shell.

In some embodiments the anti-cancer agent is an antibody or a functional antibody fragment, or a proteinaceous binding molecule with antibody-like functions. Examples of (recombinant) antibody fragments are Fab fragments, Fv fragments, single-chain Fv fragments (scFv), diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). Single-chain Fv fragments are for instance fusions of variable regions from one heavy chain and one light chain of an immunoglobulin molecule. An example of a proteinaceous binding molecule with antibody-like functions is a mutein based on a polypeptide of the lipocalin family (WO 2003/029462; WO 2005/019254; WO 2005/019255; WO 2005/019256; Beste et al., Proc. Natl. Acad. Sci. USA (1999) 96, 1898-1903). Lipocalins, such as the bilin binding protein, the human neutrophil gelatinase-associated lipocalin, human Apolipoprotein D, human tear lipocalin, or glycodelin, posses natural ligand-binding sites that can be modified so that they bind to selected small protein regions known as haptens. Other non-limiting examples of further proteinaceous binding molecules so-called glubodies (see WO 96/23879), proteins based on the ankyrin scaffold (Mosavi, L. K., et al., Protein Science (2004) 13, 6, 1435-1448) or the crystalline scaffold (WO 2001/04144), the proteins described by Skerra (J. Mol. Recognit. (2000) 13, 167-187), AdNectins, tetranectins, avimers and peptoids. Avimers contain so called A-domains that occur as strings of multiple domains in several cell surface receptors (Silverman, J, et al., Nature Biotechnology (2005) 23, 1556-1561). Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding (ibid.). Peptoids, which can act as protein ligands, are oligo(N-alkyl) glycines that differ from peptides in that the side chain is connected to the amide nitrogen rather than the α carbon atom. Peptoids are typically resistant to proteases and other modifying enzymes and can have a much higher cell permeability than peptides (see e.g. Kwon, Y.-U., and Kodadek, T., J. Am. Chem. Soc. (2007) 129, 1508-1509). Where desired, a modifying agent may be used that further increases the affinity of the respective moiety for any or a certain form, class etc. of target matter.

In some embodiments the anti-cancer agent is an anthracycline compound, such as Daunorubicin, Doxorubicin, Epirubicin or Idarubicin. In some embodiments the anti-cancer agent is an alkylating agent such as Bendamustine, Carboplatin, Cispltin, Cyclophoshamide, Estramustine, Itosfamde, Mechlorethamine, Melphalan or Oxaliplatin.

In some embodiments the anti-cancer agent is or includes a molecule that inhibits the function of protein kinases. Some small organic molecules form a class of compounds that modulate the function of protein kinases. Examples of molecules that have been reported to inhibit the function of protein kinases include, but are not limited to, his monocyclic, bicyclic or heterocyclic aryl compounds (PCT WO 92/20642, published Nov. 26, 1992 by Maguire et al.), vinylene-azaindole derivatives (PCT WO 94/14808, published Jul. 7, 1994 by Ballinari et al.), 1-cyclopropyl-4-pyridyl-quinolones (U.S. Pat. No. 5,330,992), styryl compounds (U.S. Pat. No. 5,217,999), styryl-substituted pyridyl compounds (U.S. Pat. No. 5,302,606), certain quinazoline derivatives (EP Application No. 0 566 266 A1), seleoindoles and selenides (PCT WO 94/03427, published Feb. 17, 1994 by Denny et al.), tricyclic polyhydroxylic compounds (PCT WO 92/21660, published Dec. 10, 1992 by Dow), and benzylphosphonic acid compounds (PCT WO 91/15495, published Oct. 17, 1991 by Dow et al). As a few illustrative examples of tyrosine kinase inhibitors may serve here Dasatinib, Erlotinib, Gefitinib, Imatinib, Lapatinib, Nilotinib, Sorafenib and Sunitinib.

Compounds that would be able to traverse cell membranes and are resistant to acid hydrolysis are potentially advantageous as therapeutics as they can become highly bio-available after being administered orally to patients. However, such protein kinase inhibitors typically only weakly inhibit the function of protein kinases. In addition, many inhibit a variety of protein kinases and will cause multiple side-effects as therapeutics for diseases. Accordingly, the use of microparticles of the present invention overcomes this draw-back by providing a passage into cancer cells for compounds that cannot cross the cell membrane.

Furthermore, typical routes of take-up of particles in cells, e.g. via endocytosis, guide the internalized particles to closed vesicles such as endosomes or phagosomes. Such vesicles subsequently become fused with lysosomes, which often leads to a rapid destruction of therapeutic molecules with little release into the cytosol. There is however no indication that in cancer cells microparticles of the invention end up in acidified vesicles, where they would quickly become degraded. Hence, the use of microparticles of the invention allows for the delivery and, if desired, a slow release of a pharmaceutically active compound such as an anti-cancer agent and of a nanoparticle to the interior, including the cytosol, of a cancer cell. As an example, PLGA with a half life of about 2 months allows, when used for the formation of a microparticle of the invention, for a slow release over an extended period of time.

Other examples of substances capable of modulating kinase activity include, but are not limited to, indolinones, tyrphostins, quinazolines, quinoxolines, and quinolines. The indolinones, quinazolines, tyrphostins, quinolines, and quinoxolines referred to above include well known compounds such as those described in the literature.

In some embodiments the anti-cancer agent is present in the form of a pharmaceutically acceptable salt. The term “pharmaceutically acceptable salt” refers to salts of the parent compound that do not, or at least not significantly or adversely, affect the biocompatibility, such as pharmaceutical side effects or toxicity, of the parent compound. Examples of suitable salts include chlorides, iodides, bromides, hydrochlorides, acetates, nitrates, stearates, phosphates or sulfates.

In some embodiments the anti-cancer agent is a nucleic acid molecule, such as DNA or RNA. In some embodiments the anti-cancer agent is a non-coding nucleic acid molecule, such as for example an aptamer or a Spiegelmer® (described in WO 01/92655). A non-coding nucleic acid molecule may also be an nc-RNA molecule (see e.g. Costa, F F, Gene (2005), 357, 83-94 for an introduction on natural nc-RNA molecules). Examples of nc-RNA molecules include, but are not limited to, an anti-sense-RNA molecule, an L-RNA Spiegelmer®, a silencer-RNA molecule (such as the double-stranded Neuron Restrictive Silencer Element), a micro RNA (miRNA) molecule, a short hairpin RNA (shRNA) molecule, a small interfering RNA (siRNA) molecule, a repeat-associated small interfering RNA (rasiRNA) molecule or an RNA that interacts with Piwi proteins (piRNA) (for a brief review see e.g. Lin, H., Science (2007) 316, 397).

In some embodiments the anti-cancer agent is a nucleosid analog. Nucleosid analogs are generally exposed to complex transformation after administration until they are converted to nucleoside analog 5′-triphosphates that terminate cellular transcription and DNA replication. Tests and trials on many prospective nucleosid analogs had to be stopped or discarded early as too high doses had to be applied to achieve desired intracellular conversion to nucleoside analog 5′-triphosphates. The microparticles of the invention allow the selective delivery of such nucleoside analogs into cancer cells and thus allow their use without high doses of the corresponding free compounds. Gemcitabine (2′,2′-difluorodeoxycytidine), Cladribine, Fludarabine, Clofarabine, Mercaptopurine, Telbivudine, 2-CDA, 5-Fluorouracil, AZT, Pentostatin, Azacitidine, Capecitabine, Cytarabine, Decitabine and Gemcitabine are suitable examples of nucleoside analogs.

In some embodiments the anti-cancer agent is an antimicrotubule agent such as a Vinca alkaloid, e.g. Vinblastine, Vincristine or Vinorelbine, or a Taxane, e.g. Docetaxel or Paclitaxel. A further example of an antimicrotubule agent is Ixabepilone. Further examples of an anti-cancer agent include, but are not limited to, arsenic trioxide, Altretamine, Bleomycin, Bortezomib, Dactinomycin, Denileukin, hydroxyurea, Lenalidomide, Mitomycin, Mitoxantrone, Temsirolimus, Thalidomide or Tretinoin.

In another aspect, the invention provides methods for treating or evaluating cancer by administering to cells of an organism, including a mammal such as a human, in need of such treatment an anti-cancer agent. Any of these methods is based on the use of one or more microparticles of a size in the range from about 2 to about 5 μm, such as about 3 to about 5 μm, of about 3.5 to about 5 μm, or about 4 to 5 μm. As explained above, where a plurality of microparticles is used, a respective plurality of microparticles may in some embodiments include microparticles of any width within a range as named above. The widths of the microparticles may be uniformly or non-uniformly distributed within such a width range. In some embodiments the microparticles may be of, or at least essentially of, the same width. As a few illustrative examples, all or at least essentially all microparticles may be of a width of about 2 μm, of about 2.5 μm, of about 3 μm, of about 3.5 μm, of about 4 μm, of about 4.5 μm or of about 5 μm. Any technique available in the art can be employed to obtain microparticles of a desired width or of a desired width range, such as for example size-selective fractionation.

The microparticles used in a method of the invention further include at least one of an anti-cancer agent (herein also termed the “drug”) and one or more nanoparticles, e.g. nanocrystals. In some embodiments the microparticles include both a nanoparticle and an anti-cancer agent. The above explanations and definitions apply mutatis mutandis with the proviso that a microparticle used in a method of the invention may in some embodiments include only a nanoparticle, e.g. nanocrystal, or only an anti-cancer agent, but not necessarily both. Further a microparticle used in a method of the invention includes a biocompatible polymer as defined and explained above.

The anti-cancer agent may accordingly be included in a microparticle as defined above. The present inventors have made the surprising finding that microparticles of about 2 μm to about 5 μm are preferably taken up by cancer cells, while they are hardly, if at all taken up by non-cancer cells, i.e. healthy cells. Only phagocyting cells of the body, generally macrophages, can otherwise be expected to take up microparticles of this size. Differences between cancer cells and non-cancer cells in cellular uptake have previously not been expected and accordingly not been investigated. There had merely been an isolated report that MCF-7 and BT474 human breast cancer cells take up more silica nanoparticles of a width of 150 nm than MVF-10A non-cancer cells (abstract of Iwakuma, N., et al., Nanotech 2007, Technical Proceedings of the 2007 NSTI Nanotechnology Conference and Trade Show, Vol. 2, 348-351). Only after the priority date of the present application further evidence has been published that points to a different endocytic pathway between cancer cells and non-cancer cells (abstract of Sahay, G., et al., Biomaterials (2009) doi:10.1016/j.biomaterials. 2009.09.101).

Larger particles are more desirable as they can load a range of payload molecules including drugs, DNA, enzymes, and imaging tags and release greater amounts of medicine over a longer period of time. Furthermore, microparticles have larger available surface area of contact with the cells and hence higher binding to the cells. This may further increase the chance of being internalized into the cells, with a local release of the drug. Besides, tumor vasculatures are known to be leaky. Therefore microparticles are able to reach tumor tissue despite their relatively large size.

A preferred or a selective uptake by cancer cells (vs. non-cancer cells) provides the further advantage of relatively low toxicity to normal cells, increased efficacy of the drug delivery system, as well as the possibility to increase drug dosage with decreased toxicity or systemic side effects.

The microparticles may be administered to the corresponding organism or to a tissue sample thereof. By administering the microparticles to a tissue sample cancer in the organism may be evaluated, for example by means of microscopy. The uptake of the microparticles and the response of the cells of a respective tissue sample may be monitored, for example in order to predict the success of an intended treatment with drug loaded microparticles of the invention. Likewise, cancer therapy by means of such microparticles may be monitored, due to the presence of detectable QDs in the microparticles. Thus, the present invention also encompasses a method that allows predicting or diagnosing cancer in a subject. The method includes the steps of (a) obtaining a biological sample from the subject; and (b) exposing the sample to a plurality of microparticles as defined above. The method typically also includes detecting the microparticles in said sample.

Accordingly, one or more microparticles may also be used in the manufacture of a medicament for the treatment of cancer. Such microparticles include a biocompatible polymer and an anti-cancer agent, as described above. The microparticles generally have a width of at least about 1 μm (see above). The microparticles may also include one or more nanoparticles (see above). Respective microparticles may further be used in an in vitro or in an ex-vivo method, for example for testing or screening purposes.

A further use of respective microparticles is a method of selectively directing an anti-cancer agent, or a pharmaceutically acceptable salt, ester, prodrug or hydrate thereof, to one or more cancer cells within a plurality of healthy cells. The method may be a method of selectively directing an anti-cancer agent, or a pharmaceutically acceptable salt, ester, prodrug or hydrate thereof, to cancer cells that are included in a multicellular organism. In some embodiments the method is an ex-vivo or an in vitro method. It may for example be a method of screening candidate compounds. In such a method a cell population may be used that includes both healthy cells and cancer cells. Such cell population may include at least one cancer cell. The cell population may then be contacted with a respective microparticle, i.e. microparticle that includes a biocompatible polymer and an anti-cancer agent or a pharmaceutically acceptable salt, ester, prodrug or hydrate thereof. If the microparticles include one or more nanoparticles, these nanoparticles may be used for monitoring and detection (supra).

The term “prodrug” as used herein means a compound or a pharmaceutically acceptable salt thereof that is convertible in vivo metabolically or by solvolysis into a physiologically active anti-cancer agent. The prodrug itself may or may not also have activity with respect to a target cancer cell. Prodrugs include, without limitation, esters, amides, carbamates, carbonates, ureides, or solvates of the active anti-cancer agent. For example, a compound with a hydroxy group may be provided in a microparticle as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoates, gentisates, isethionates, di-p-toluoyltartrates, methane-sulfonates, ethanesulfonates, benzenesulfonates, p-toluenesulfonates, cyclohexylsulfamates, quinates, esters of amino acids, to name a few. Similarly, a compound that has an amino group may be provided in a microparticle as an amide that is converted by hydrolysis in vivo to the amine compound. Typically, the prodrug is inactive, or less active than the active compound, but may provide one or more advantageous handling, administration, and/or metabolic properties. Some prodrugs are activated enzymatically to yield the active anti-cancer agent, or a compound may undergo further chemical reaction to yield the active anti-cancer agent. A prodrug may proceed from prodrug form to active form in a single step or may have one or more intermediate forms which may themselves have activity or may be inactive.

The present invention also provides a method for screening for cancer cells for their responsiveness to a selected compound. The method allows the selective application of such compound to the cancer cells in presence of healthy non-cancer cells. The method may involve identifying a mutant polypeptide in human cells using techniques that are routine and standard in the art, such as those described herein for identifying mutant kinases (e.g., cloning, Southern or Northern blot analysis, in situ hybridization, PCR amplification, etc.).

In this regard the invention further provides a method of identifying a cell that is resistant to apoptosis inducing reagents, i.e. a cell that is chemoresistant. In the method a cell is exposed to a microparticle of a width of about 2-5 μm, carrying an apoptosis inducing reagent. The effect of the reagent released in the cell can then be monitored.

Microparticles according to the invention may be formed by any technique available in the art, including a technique based on an emulsion, such as suspension polymerization, solvent evaporation, organic phase separation (coacervation) and spray-drying.

As an illustrative example, an emulsion may be formed from any suitable combination of immiscible liquids. For this purpose a polar phase and a non-polar phase may be provided by providing a respective polar solvent and a non-polar solvent. A respective polar solvent may be any dipolar solvent, for example, a dipolar aprotic or dipolar protic solvent. In some embodiments the nanoparticles and the biocompatible polymer are provided in a non-polar solvent. Where the anti-cancer agent is a non-polar compound it can simply be added in the desired concentration to the non-polar phase. As an immiscible liquid an aqueous solution may be employed, thereby forming an oil-in-water emulsion. Where the anti-cancer agent is a polar compound it can be added in the desired concentration to this polar phase.

Examples of non-polar liquids include, but are not limited to mineral oil, hexane, heptane, cyclohexane, benzene, toluene, dichloromethane, chloroform, carbon tetrachloride, carbon disulfide, dioxane, diethyl ether, diisopropylether, methyl propyl ketone, methyl isoamyl ketone, methyl isobutyl ketone, cyclohexanone, isobutyl isobutyrate, ethylene glycol diacetate, and a non-polar ionic liquid. Examples of a non-polar ionic liquid include, but are not limited to, 1-ethyl-3-methylimidazolium bis[(trifluoromethyl)sulfonyl]amide bis(triflyl)-amide, 1-ethyl-3-methylimidazolium bis[(trifluoromethyl)sulfonyl]amide trifluoroacetate, 1-butyl-3-methylimidazolium hexafluorophosphate, 1-hexyl-3-methylimidazolium bis(tri-fluoromethylsulfonyl)imide, 1-butyl-3-methylimidazolium bis(trifluoromethylsulfonyl)imide, trihexyl(tetradecyl)phosphonium bis[oxalato(2-)]borate, 1-hexyl-3-methyl imidazolium tris-(pentafluoroethyl)trifluorophosphate, 1-butyl-3-methyl-imidazolium hexafluorophosphate, tris(pentafluoroethyl)trifluorophosphate, trihexyl(tetradecyl)phosphonium, N″-ethyl-N,N,N′, N′-tetramethylguanidinium, 1-butyl-1-methylpyrrolidinium tris(pentafluoro ethyl)trifluoro-phosphate, 1-butyl-1-methylpyrrolidinium bis(trifluoromethylsulfonyl) imide, 1-butyl-3-methyl imidazolium hexafluorophosphate, 1-ethyl-3-methylimidazolium bis(trifluoromethyl-sulfonyl)imide and 1-n-butyl-3-methylimidazolium.

Examples of dipolar aprotic liquids are methyl ethyl ketone, chloroform, tetrahydrofuran, dioxane, ethylene glycol monobutyl ether, pyridine, methyl isobutyl ketone, acetone, cyclohexanone, ethyl acetate, isobutyl isobutyrate, ethylene glycol diacetate, dimethylformamide, acetonitrile, N,N-dimethyl acetamide, nitromethane, acetonitrile, N-methylpyrrolidone, methanol, ethanol, propanol, isopropanol, butanol, N,N-diisopropylethyl-amine, and dimethylsulfoxide. Examples of polar protic solvents include, but are not limited to methanol, ethanol, butyl alcohol, formic acid, dimethylarsinic acid [(CH₃)₂AsO(OH)], N,N-dimethyl-formamide, N,N-diisopropylethylamine, or chlorophenol.

In typical embodiments of such a method a biocompatible polymer and, where the microparticle to be formed includes a nanoparticle, a nanoparticle are provided. In the process a polar solvent and a non-polar solvent are provided. The polar solvent may, for example, be water or an aqueous solution. The polar solvent and the non-polar solvent are at least substantially immiscible with each other. The biocompatible polymer is dissolved in one of the polar solvent and the non-polar solvent. Thus, in some embodiments where the polymer is hydrophilic it may be dissolved in the polar solvent. The nanoparticle is dispersed in one of the polar solvent and the non-polar solvent. Where the nanoparticle is a polar nanoparticle it may, for example, be dispersed in the polar solvent. Where the nanoparticle is a non-polar nanoparticle it may, for example, be dispersed in the non-polar solvent. In some embodiments the nanoparticle and the biocompatible polymer are dissolved/dispersed in the same solvent. In some embodiments the nanoparticle and the biocompatible polymer are dissolved/dispersed in the same solvent. In some embodiments the nanoparticle is dissolved in a solvent that differs from the solvent in which the biocompatible polymer is dispersed. The non-polar solvent and the polar solvent are combined, so that a two-phase mixture is formed. The two-phase mixture is agitated, for example stirred. The formation of a microparticle is thereby allowed. In some embodiments the process of the formation of the microparticle includes emulsification. A homogenizer may be used in the process of the invention. Agitation can involve external movement such as shaking, for instance of a container encompassing the mixture, or internal fluid circulation. As an illustrative example, mechanical agitation; e.g. mechanical mixing such as rotary mixing, may be employed. Any desired means may be used to carry out agitation. As an example, a stirrer in the form of any commercially available stirrer with any maximal stirring speed may be used, as long as a desired stirring speed can be achieved, resulting in a desired intensity of agitation. Illustrative examples of suitable stirrers may for instance have a range of stirring speeds from 0-about 500 rpm, 0-about 1000 rpm, 0-about 1800 rpm or 0-about 5000 rpm.

The position of the nanoparticle within the microparticle can be controlled by the polarity of the nanoparticle. Further, the position of the nanoparticle within the microparticle can be controlled by the ratio of the volume of the non-polar solvent to the polar solvent used. The more the polarity of the nanoparticle differs from the polarity of the biocompatible polymer and the more it corresponds to the polarity of the solvent used in excess the more will the nanoparticle be positioned in proximity to the surface of the microparticle. Hence, typically the position of the nanoparticle in the final microparticle depends on the hydrophobicity or polarity of the nanoparticle. Where a polar nanoparticle is used, an increase in the difference in polarity between the nanoparticle and the biocompatible polymer positions the nanoparticle closer to the surface of the microparticle. Where a non-polar nanoparticle is used, a decrease in the difference in polarity between the nanoparticle and the biocompatible polymer positions the nanoparticle closer to the surface of the microparticle.

In some embodiments, for instance, a non-polar nanoparticle is used, as well as a polar polymer. Further an excess of non-polar solvent may be employed. In such a case the nanoparticle will be positioned closer to the surface of the microparticle the more non-polar solvent is used in relation to the polar solvent. In some embodiments, for instance, a polar nanoparticle is used, as well as a polar polymer. In such a case the nanoparticle will be positioned randomly within the microparticle. If in such a case a plurality of nanoparticles is used they will be distributed uniformly across the microparticle. The position of the nanoparticles within the microparticle may also be controlled by the intensity of agitation, e.g. the speed of stirring. An increase in the intensity of agitation positions the nanoparticle closer to the surface of the microparticle.

The non-polar solvent and the polar solvent may be used in any desired ratio. In some embodiments the polar solvent is used in excess when compared to the non-polar solvent. In some embodiments the ratio of the non-polar solvent to the polar solvent used is selected to be in the range from about 1:20 to about 1:1, such as about 1:10 to about 1:1, about 1:10 to about 1:2, about 1:8 to about 1:1, about 1:8 to about 1:2, about 1:6 to about 1:1, about 1:6 to about 1:2, about 1:6 to about 1:3, including a ratio of about 1:6.

In some embodiments the nanoparticle is a non-polar nanoparticle and the biocompatible polymer is a polar or an amphiphilic polymer. In such an embodiment the process of forming the microparticle may include providing a biocompatible polymer. The process includes providing a nanoparticle. Further, the process includes dissolving the biocompatible polymer and dispersing the nanoparticle in a suitable non-polar solvent. Thereby a non-polar phase is formed. The process also includes adding to the non-polar phase a polar solvent. The polar solvent is at least substantially immiscible with the non-polar solvent. By adding the polar solvent a two-phase mixture is formed. The process furthermore includes agitating the two-phase mixture. Thereby the process includes allowing the formation of a microparticle. The position of the nanoparticle within the microparticle is controlled by (i) the ratio of the volume of the non-polar phase to the polar solvent and/or by (ii) the intensity of agitation. An increase in the ratio of the volume of the non-polar phase to the polar solvent positions the nanoparticle closer to the surface of the microparticle. An increase in the intensity of agitation also positions the nanoparticle closer to the surface of the microparticle.

In some embodiments the nanoparticle is a polar nanoparticle and the biocompatible polymer is a polar polymer. In such an embodiment the process of forming the micoparticle may include providing the biocompatible polymer. The process includes providing the nanoparticle. Further, the process includes dissolving the biocompatible polymer and dispersing the nanoparticle in a suitable polar solvent. Thereby a polar phase is formed. The process also includes adding to the polar phase a non-polar solvent. The non-polar solvent is at least substantially immiscible with the polar solvent. By adding the non-polar solvent a two-phase mixture is formed. The process furthermore includes agitating the two-phase mixture. Thereby the process includes allowing the formation of a microparticle. As noted above, the position of the nanoparticle within the microparticle is controlled by the intensity of agitation. An increase in the intensity of agitation positions the nanoparticles closer to the surface of the microparticle (supra). The position of the nanoparticles within the microparticles is also controlled by the ratio of the volume of the non-polar solvent to the polar phase. An increase in the ratio of the volume of the non-polar phase to the polar solvent positions the nanoparticle closer to the centre of the microparticle.

As explained above, the microparticle may include a plurality of nanoparticles. In the formation of such a microparticle a plurality of nanoparticles may be dispersed in a suitable solvent. A plurality of non-polar nanoparticles may, for instance, be dispersed in the suitable non-polar solvent above. A plurality of polar nanoparticles may, as a further example, be dispersed in a polar solvent. The position of the nanoparticles within the microparticle is then furthermore controlled by the amount of nanoparticles dispersed (see above). A decrease in the amount of nanoparticles positions the nanoparticles closer to the surface of the microparticle, and vice versa.

Where desired, the microparticles of the invention may be designed for sustained and for controlled delivery. In a sustained system the anti-cancer agent is delivered over a prolonged period of time, which overcomes the highly periodic nature of tissue levels associated with conventional (e.g. enteral or parenteral) administration of single doses of free compounds. The term ‘controlled’ indicates in the context of administration that control is exerted over the way in which the anti-cancer agent is delivered to the tissues once it has been administrated to the organism to be treated, e.g. the patient. The use of standard biocompatible polymers is particularly useful for sustained release. Additional tissue specific ligands such as antibodies may be used for controlled release.

The microparticles described herein can be administered to an organism, including a human patient per se, or in pharmaceutical compositions where they are mixed with other active ingredients, as in combination therapy, or suitable carriers or excipient(s). Techniques for formulation and administration of respective microparticles resemble or are identical to those of low molecular weight compounds well established in the art. Exemplary routes include, but are not limited to, oral, transdermal, and parenteral delivery. A plurality of the microparticles may be used to fill a capsule or tube, or may be compressed as a pellet. The microparticles may also be used in injectable or sprayable form, for instance as a suspension or in a gel formulation.

Suitable mutes of administration may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections. It is noted in this regard that for administering microparticles a surgical procedure is not required. Where the microparticles include a biodegradable polymer there is no need for device removal after release of the anti-cancer agent. Nevertheless the microparticles may be included in or on a scaffold, a coating, a patch, composite material, a gel or a plaster.

Alternately, one may administer the microparticles in a local rather than systemic manner, for example, via injection of the compound directly into a solid tumor, often in a depot or sustained release formulation.

Pharmaceutical compositions that include the microparticles of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers including excipients and auxiliaries that facilitate processing of the microparticles into preparations that can be used pharmaceutically. Proper formulation is dependent upon the route of administration chosen.

For injection, the microparticles of the invention may be formulated in aqueous solutions, for instance in physiologically compatible buffers such as Hanks's solution, Ringer's solution, or physiological saline buffet For transmucosal administration, penetrants appropriate to the barrier to be permeated are used in the formulation. Such penetrants are generally known in the art.

For oral administration, the microparticles can be formulated readily by combining them with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatine, as well as soft, sealed capsules made of gelatine and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the microparticles may be suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

The microparticles may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions suitable for use in the present invention include compositions where the active ingredients included in the microparticles are contained in an amount effective to achieve its intended purpose. More specifically, a therapeutically effective amount means an amount of compound effective to prevent, alleviate or ameliorate cancer or prolong the survival of the subject being treated. Determination of a therapeutically effective amount is well within the capability of those skilled in the art, especially in light of the detailed disclosure provided herein.

For any compound used in the microparticles of the invention, the therapeutically effective dose can be estimated initially from cell culture assays. For example, a dose can be formulated in animal models to achieve a circulating concentration range that includes the IC50 as determined in cell culture (i.e., the concentration of the test compound which achieves a half-maximal inhibition of the kinase activity). Such information can be used to more accurately determine useful doses in humans.

The microparticles may, if desired, be presented in a pack or dispenser device which may contain one or more unit dosage forms containing the microparticles with the active ingredient. The pack may for instance include metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the compound for human or veterinary administration. Such notice, for example, may be the labeling approved by the U.S. Food and Drug Administration or other government agency for prescription drugs, or the approved product insert.

In order that the invention may be readily understood and put into practical effect, particular embodiments will now be described by way of the following non-limiting examples. It is understood that modification of detail may be made without departing from the scope of the invention.

### EXEMPLARY EMBODIMENTS OF THE INVENTION

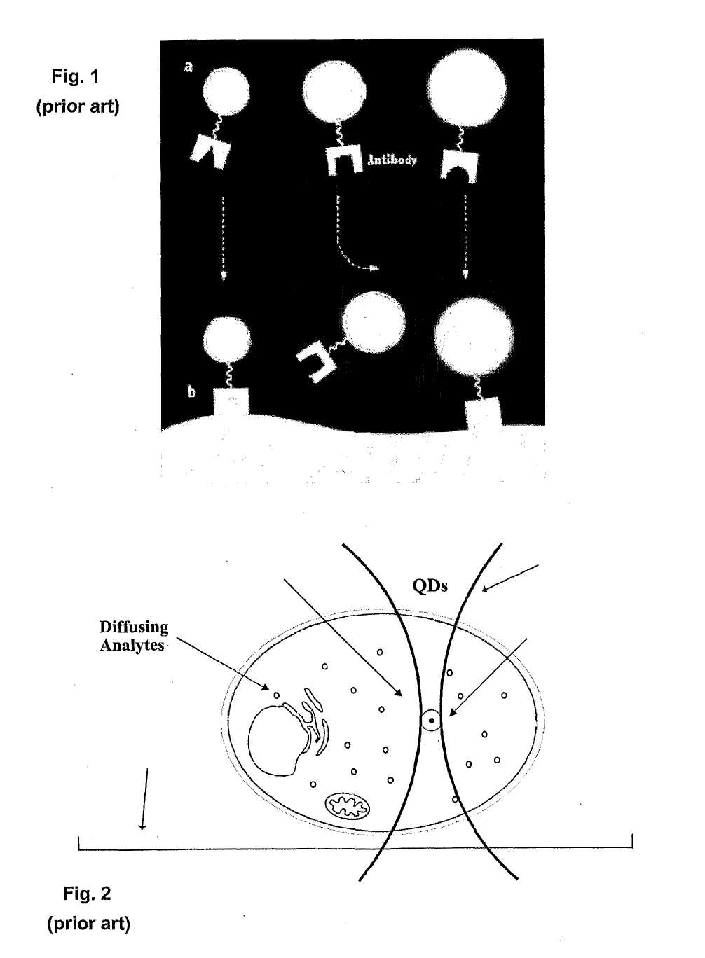

FIG. 1 and FIG. 2 illustrate the current use of quantum dots (FIG. 2) in comparison to extracellular antibodies in the art. While antibodies only reach the cell surface and are generally not an effective means to introduce compounds into a cell, quantum dots allow for internalization, a much more effective means.

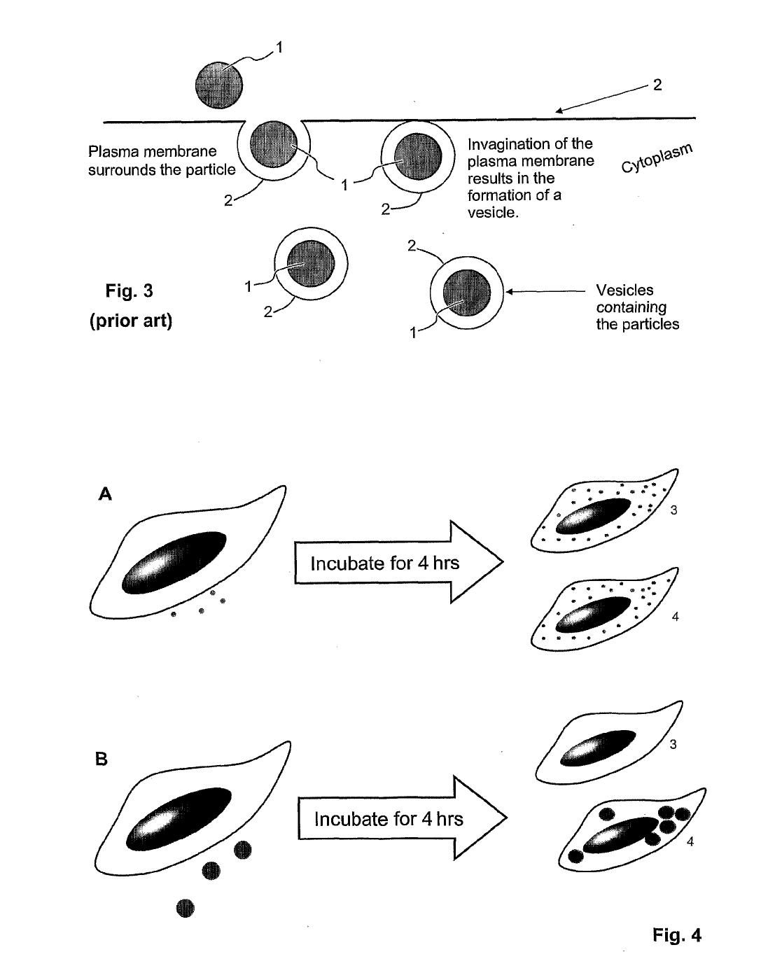

FIG. 3 illustrates the cellular uptake of quantum dots (1) via endocytosis. A quantum dot cannot diffuse across the plasma membrane. The cellular mechanism of endocytosis, however, provides a way of transportation for a quantum dot. The plasma membrane (2) thereby constricts to form a vesicle that contains the quantum dot (1).

FIG. 4 depicts the size-dependent cellular uptake of microparticles according to the invention (B) in comparison to quantum dots (A). A: Quantum dots are equally incorporated into healthy cells (3) and cancer cells (4). B: Microparticles according to the invention with a width of 2-5 μm are, within a period of 4 hrs, preferably taken up by cancer cells (4) in comparison to healthy cells (3).

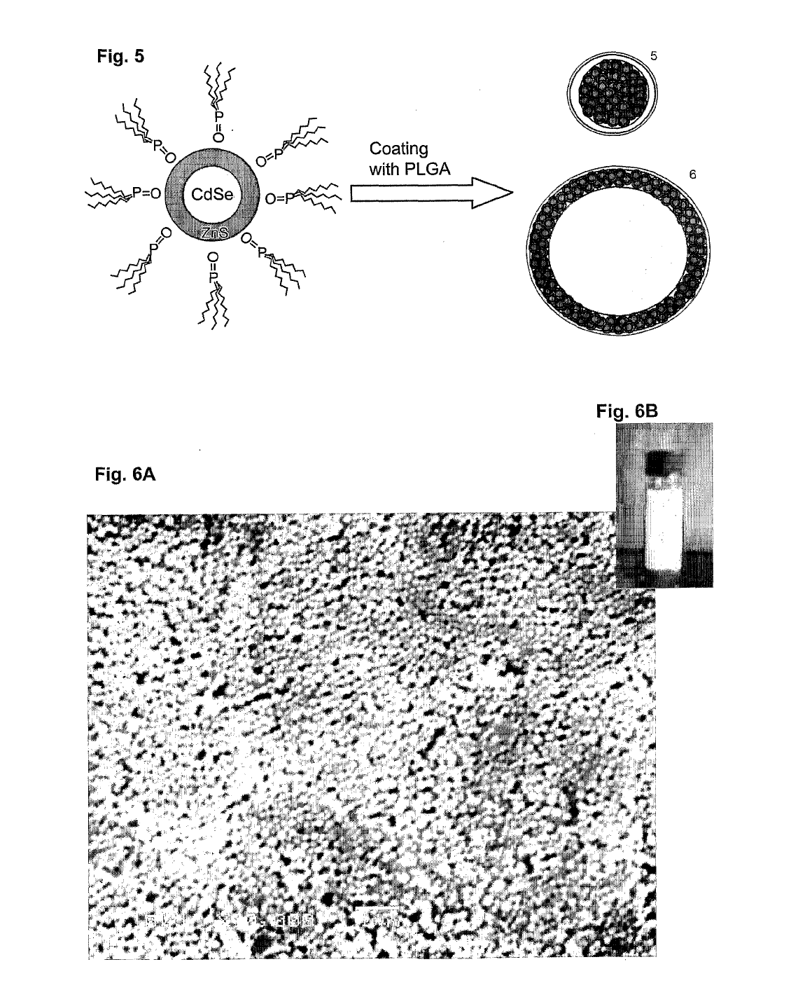

FIG. 5 depicts the formation of microparticles of the invention. QDs covered with trioctylphosphine oxide (TOPO) (left, reactant) can be incorporated into newly formed PLGA nanoparticles (5) and PLGA microparticles (6) by coating with PLGA. The latter have a visible luminescent ring-like morphology.

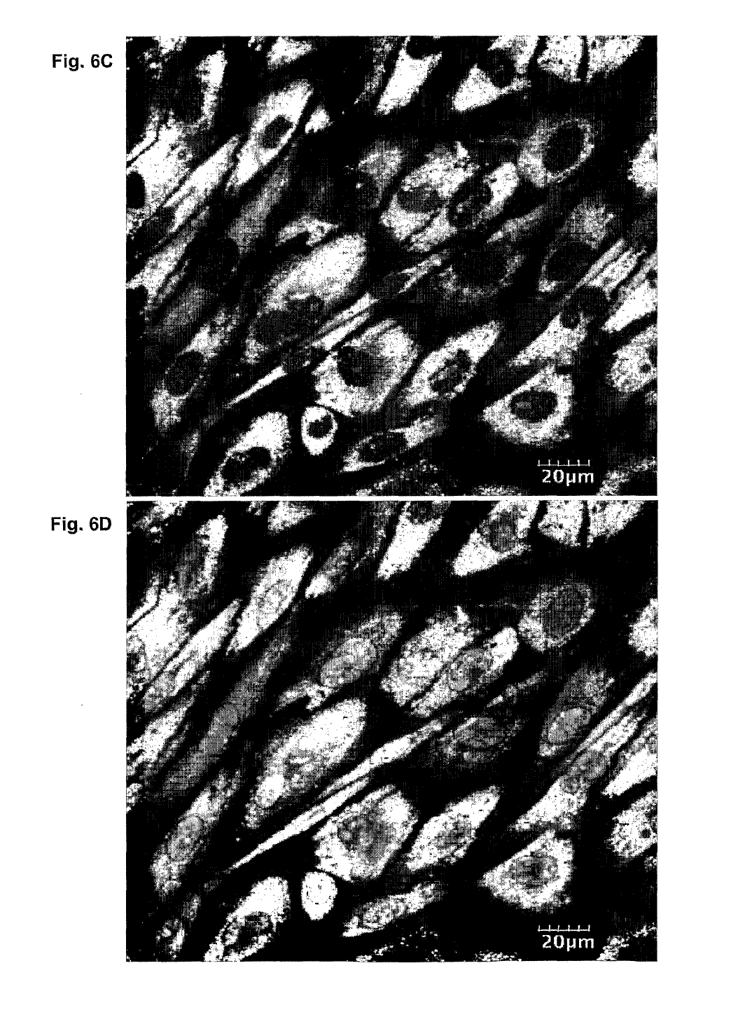

FIG. 6 shows the morphology and effective cellular uptake of luminescent PLGA nanoparticles (in comparison to the microparticles of the invention) impregnated with hydrophobic quantum dots. Monodisperse particles of 100-200 nm in size were obtained and used in these examples. FIG. 6A is a SEM image of the PLGA nanoparticles on glass substrates. FIG. 6B is a fluorescent photograph of the PLGA nanoparticles in aqueous solution under UV excitation. FIG. 6C and FIG. 6D are the same confocal image for the internalization of the green PLGA nanoparticles into the cytoplasm of CCD-112CoN cells after 2-h incubation at 37° C. followed by counterstaining of nucleus with red propidium iodide (PI). FIG. 6C is a representation of the image with only the green channel, showing the nanoparticles, while FIG. 6D is a representation of the image with all channels, thus depicting both PLGA nanoparticles and nuclei.

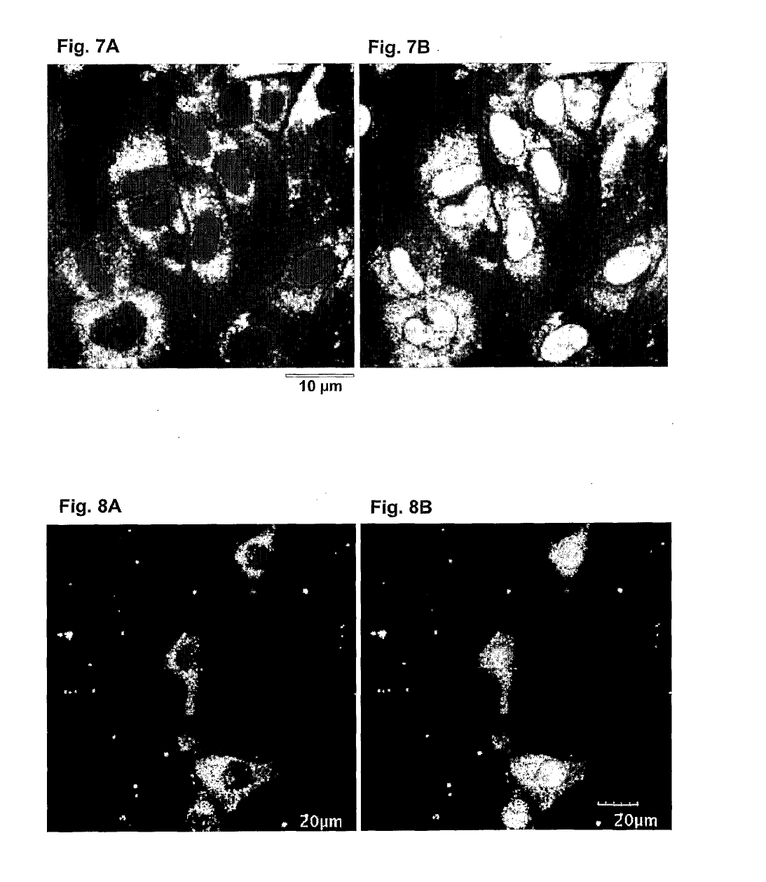

FIG. 7 shows the cellular uptake of green QD-loaded PLGA nanoparticles as universal carriers into human non-small lung cancer cells, NCI-H1299. Red PI was used for counterstaining the nucleus. FIG. 7A depicts the obtained image with only the green channel, showing the nanoparticles, while FIG. 7B depicts the obtained image with all channels, thus depicting both PLGA nanoparticles and nuclei.

FIG. 8 shows the cellular uptake of green QD-loaded PLGA nanoparticles as universal carriers into human breast cancer cells, MCF-7. Red PI was used for counter-staining the nucleus. FIG. 8A depicts the obtained image with only the green channel, showing the nanoparticles, while FIG. 8B depicts the obtained image with all channels, thus depicting both PLGA nanoparticles and nuclei.

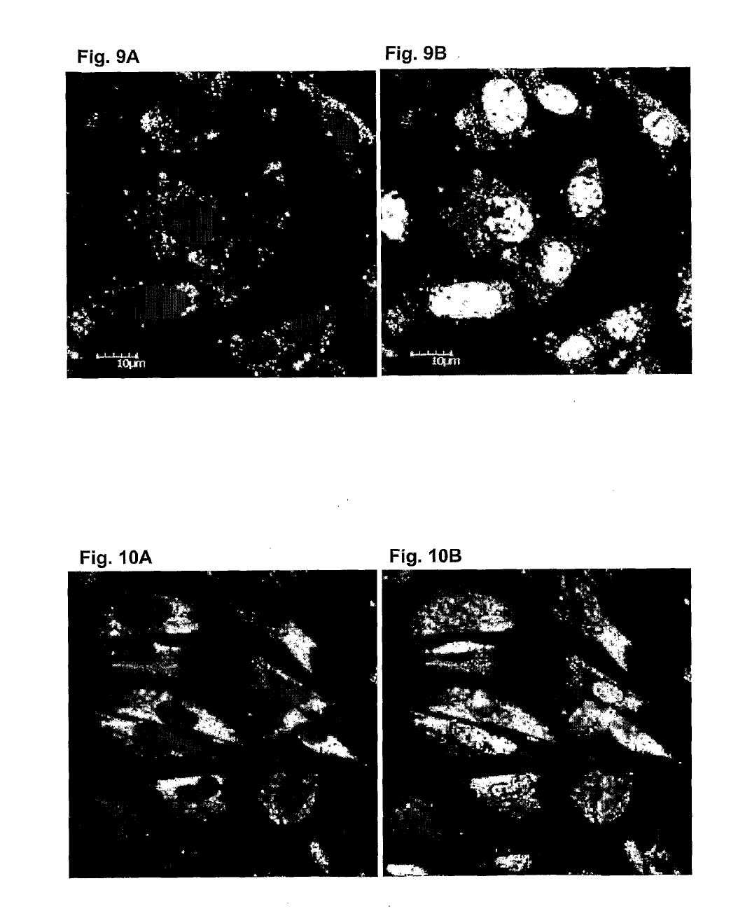

FIG. 9 shows the cellular uptake of red QD-loaded PLGA nanoparticles into rat glioma cells, C6. Blue DAPI was used for counterstaining the nucleus. FIG. 9A depicts the obtained image with only the red channel, showing the nanoparticles, while FIG. 9B depicts the obtained image with all channels, thus depicting both PLGA nanoparticles and nuclei.

FIG. 10 shows the cellular uptake of green QD-loaded PLGA nanoparticles into human normal colon cells, CCD-112CoN. Red PI was used for counterstaining the nucleus. FIG. 10A depicts the obtained image with only the green channel, showing the nanoparticles, while FIG. 10B depicts the obtained image with all channels, thus depicting both PLGA nanoparticles and nuclei.

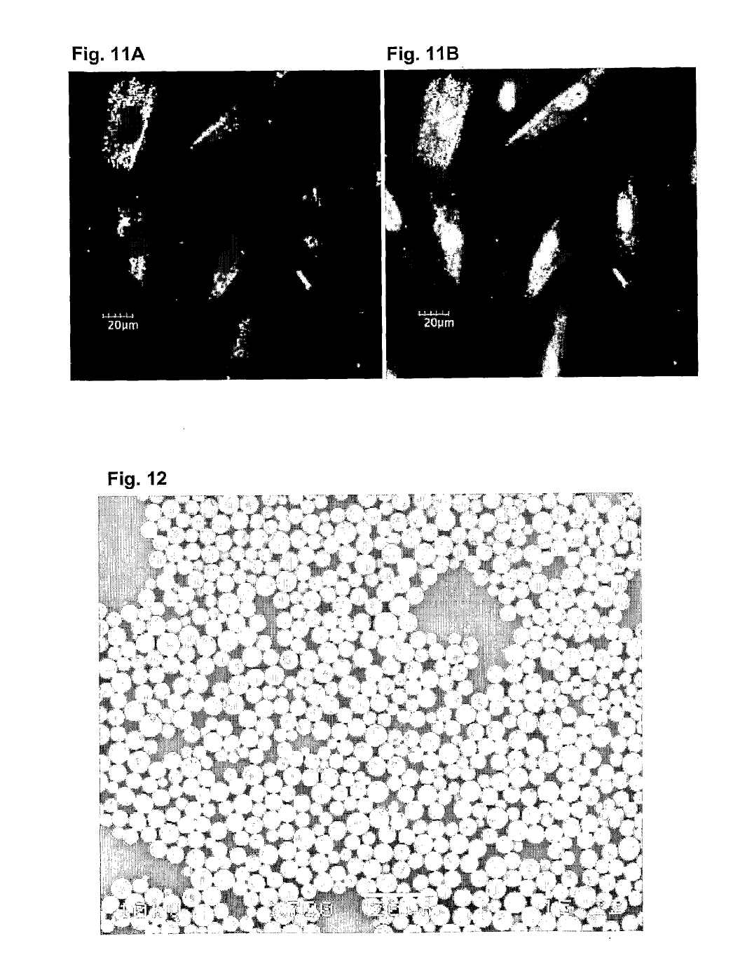

FIG. 11 shows the cellular uptake of cyan-color QD-loaded PLGA nanoparticles into Mouse fibroblasts, NIH-3T3. Blue DAPI was used for counterstaining the nucleus. FIG. 11A depicts the obtained image with only the cyan channel, showing the nanoparticles, while FIG. 11B depicts the obtained image with all channels, thus depicting both PLGA nanoparticles and nuclei.

FIG. 12-FIG. 14 depict the morphology and cellular interaction of hydrophobic QDs-incorporated microparticles according to the invention with normal cells, CCD-112CoN and breast cancer cells, MCF-7.

FIG. 12 shows an SEM image of nearly uniform microparticles after size separation.

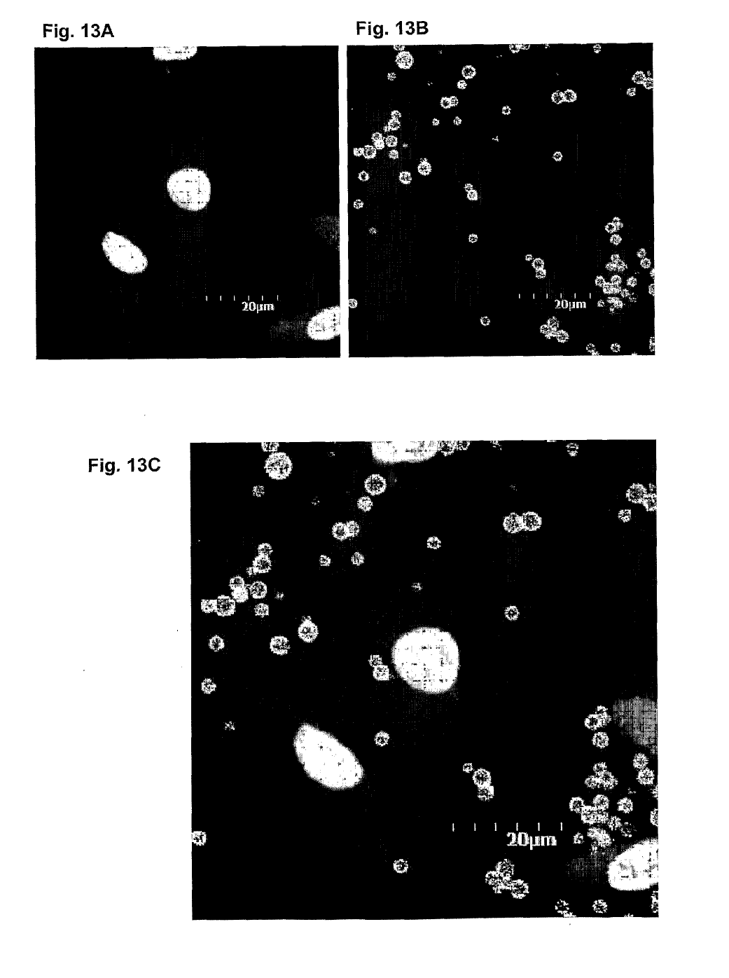

FIG. 13 shows a confocal image of normal cells after 2-h incubation at 37° C. followed by counterstaining of the nucleus with red PI, which shows limited (if any) cellular interaction with green microparticles. FIG. 13A depicts the obtained image with only the red channel, showing nuclei. FIG. 13B depicts the obtained image with only the green channel, showing the microparticles. FIG. 13C depicts the obtained image with all channels, thus depicting both PLGA microparticles and nuclei.

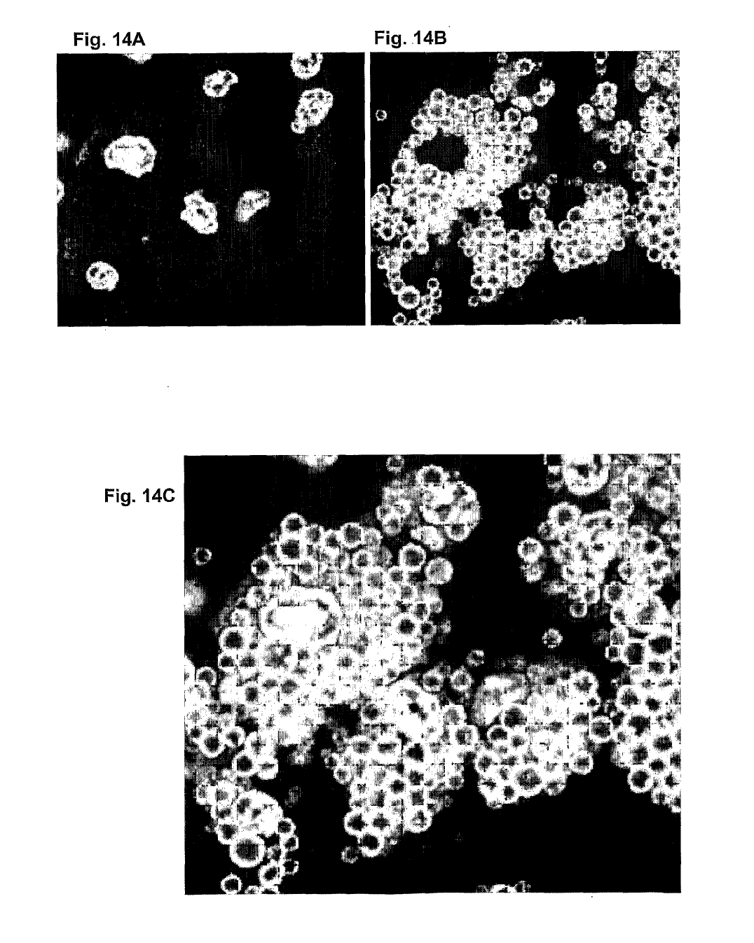

FIG. 14 shows a confocal image of breast cancer cells after 2-h incubation at 37° C. followed by counterstaining of nucleus by red PI. FIG. 14A depicts the obtained image with only the red channel, showing nuclei. FIG. 14B depicts the obtained image with only the green channel, showing the quantum dots in the microparticles. FIG. 14C depicts the obtained image with all channels, thus depicting both PLGA microparticles and nuclei. Effective cellular uptake of green microparticles formed according to the present invention can be seen.

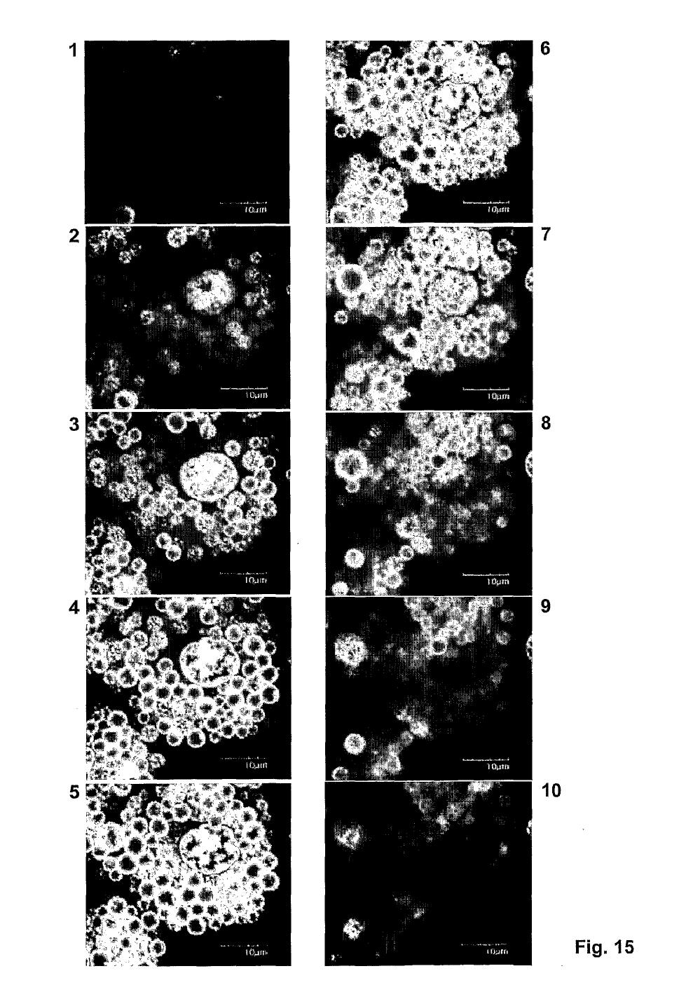

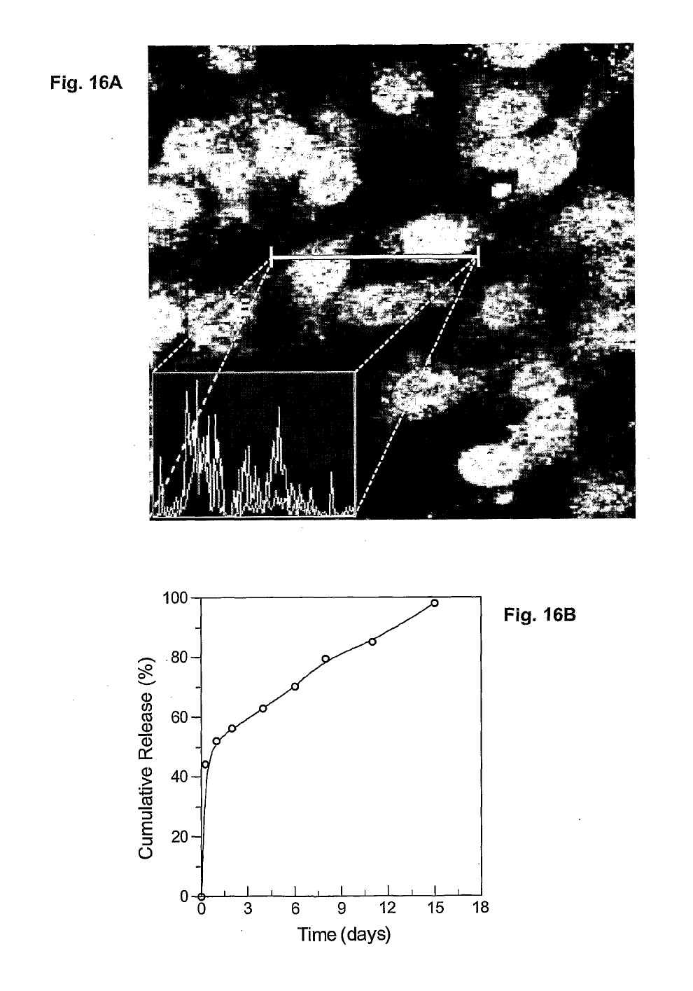

FIG. 15 illustrates intracellular distribution of microparticles by a series of confocal images of MCF-7 cell sectioned at different XY planes with 1 μm interval steps along the Z direction, after cells were incubated with green QD-incorporated PLGA microparticles and cell nuclei counterstained red with PI (all channels depicted, cf. e.g. FIG. 14).

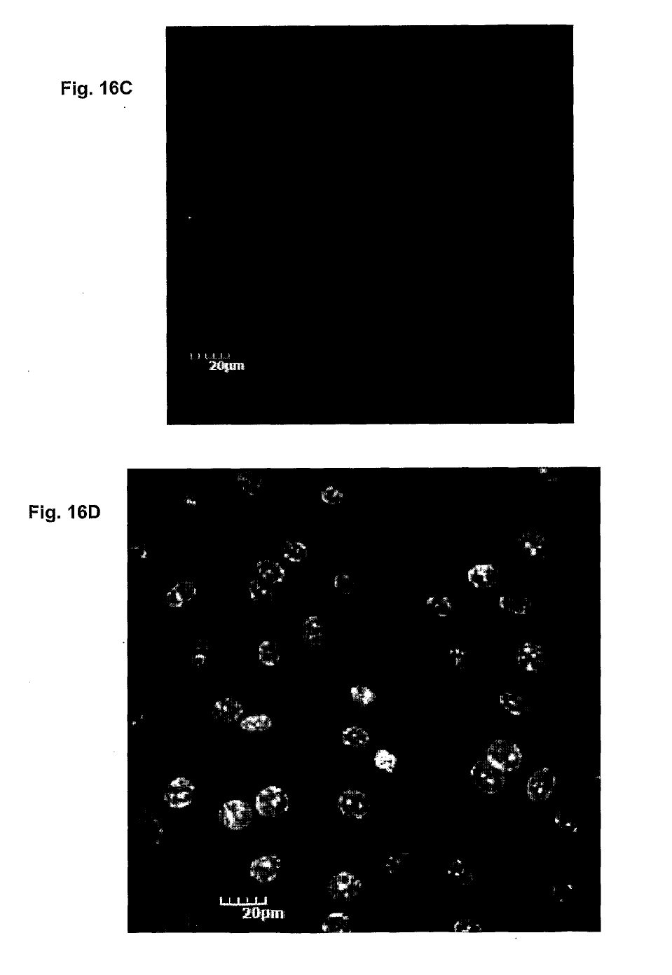

FIG. 16 depicts the in vitro uptake and controlled release behaviours of nanoparticles with Doxorubicin (DOX) incorporated therein, in comparison with the clinical dosage form of DOX. FIG. 16A is a confocal image of CCD-112CoN cells after 4-h incubation with DOX-nanoparticles at 37° C. The inset in FIG. 16A represents fluorescence intensity profiles of DOX (red, upper curve) and DAPI (blue, lower curve) in the counter-stained CCD-112CoN cells with DAPI. FIG. 16B shows the cumulative release of DOX from the DOX-nanoparticles in pH 7.4 PBS buffer solution at 37° C. FIG. 16C and FIG. 16D show confocal images of CCD-112CoN cells after 4-h incubation with DOX in the amounts after 4-h release (FIG. 16C) and complete release from the DOX-nanoparticles (FIG. 16D), respectively.

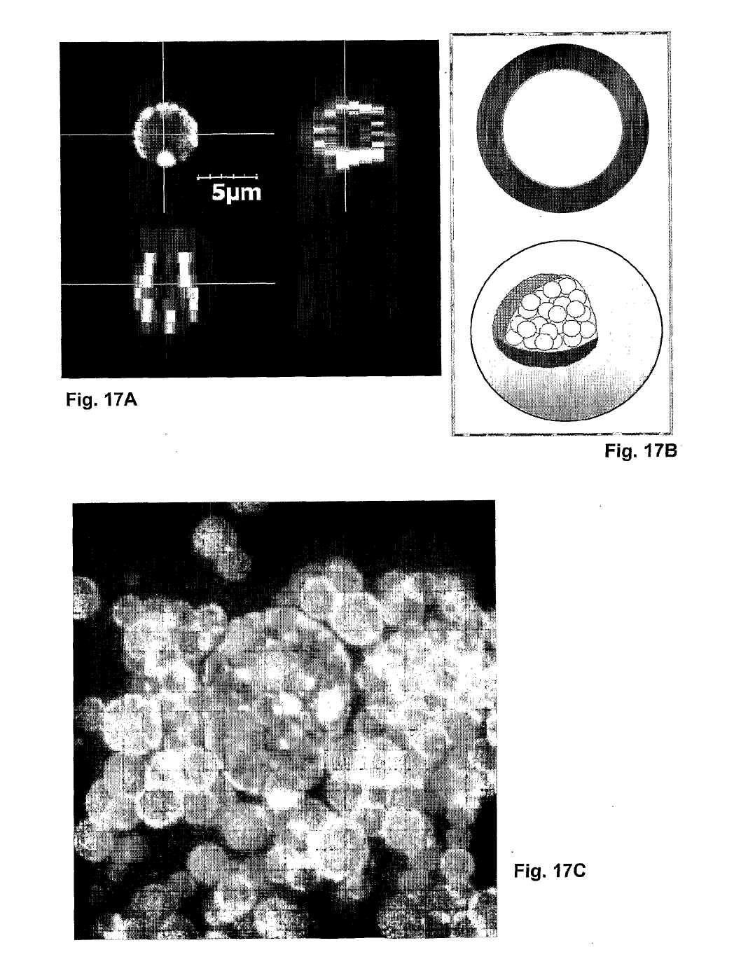

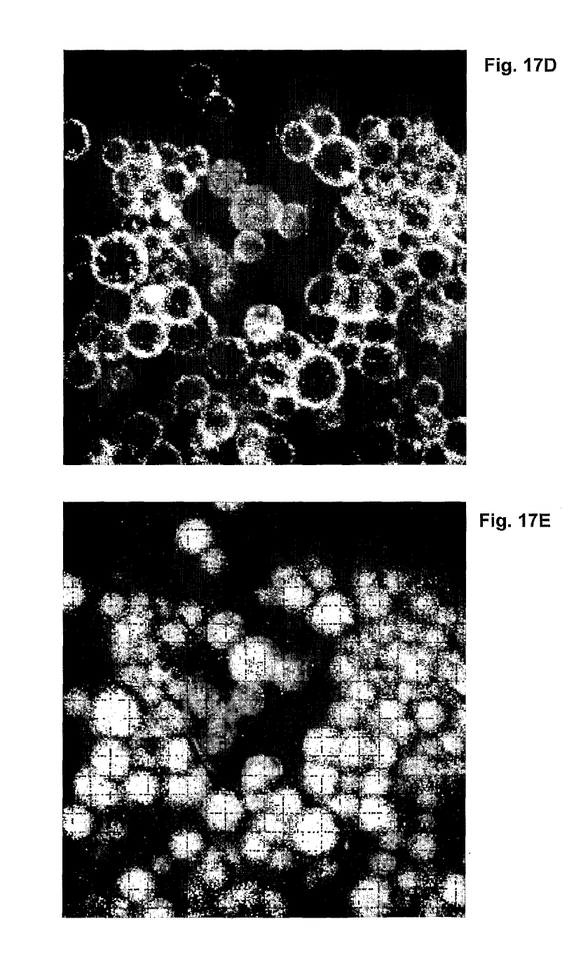

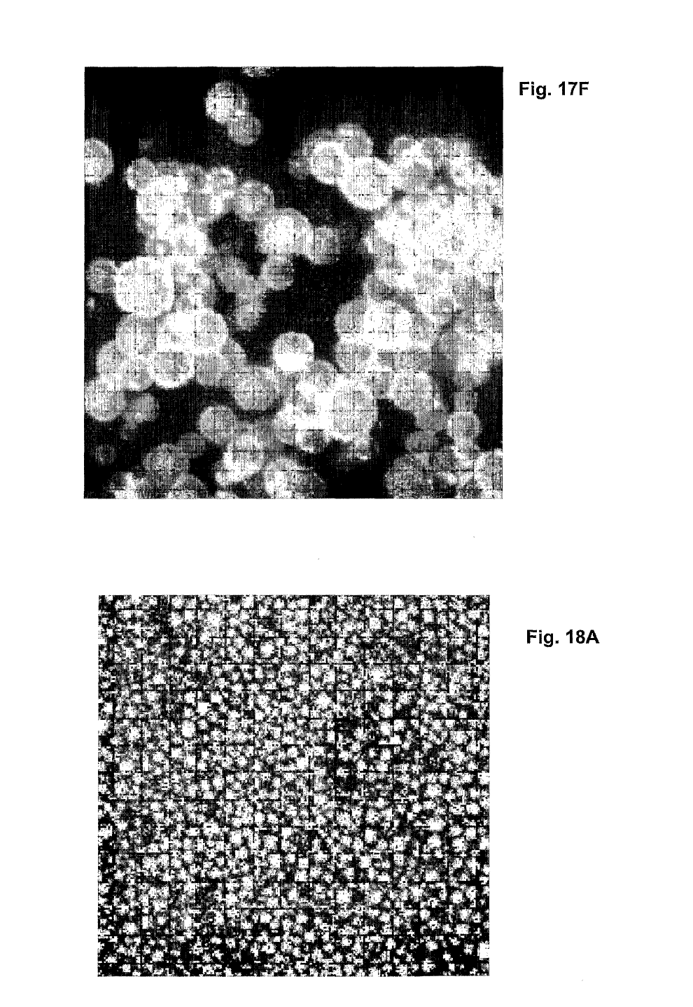

FIG. 17A is a confocal image for cross-section (in XY plane, top left image) of a green QD-incorporated PLGA microparticle of the invention in the XZ (bottom left image) and YZ (top right image) plane. FIG. 17B is a schematic illustration of a QD- and drug-loaded microparticle that indicates where QD and drug are located inside the microparticle. FIG. 17C is a confocal image of MCF-7 cell after incubation with blue drug- and green QD-incorporated PLGA microparticles. The cell nuclei were stained red with PI. All channels are shown, thus depicting PLGA microparticles, with quantum dots and drug, and nuclei. FIG. 17D depicts the obtained image with only the green channel, showing the quantum dots in the microparticles. FIG. 17E depicts the obtained image with only the blue channel, showing the drug in the microparticles. FIG. 17F depicts the obtained image with green and blue channels, thus depicting both quantum dots and drug in the microparticles (but not the cellular nuclei).

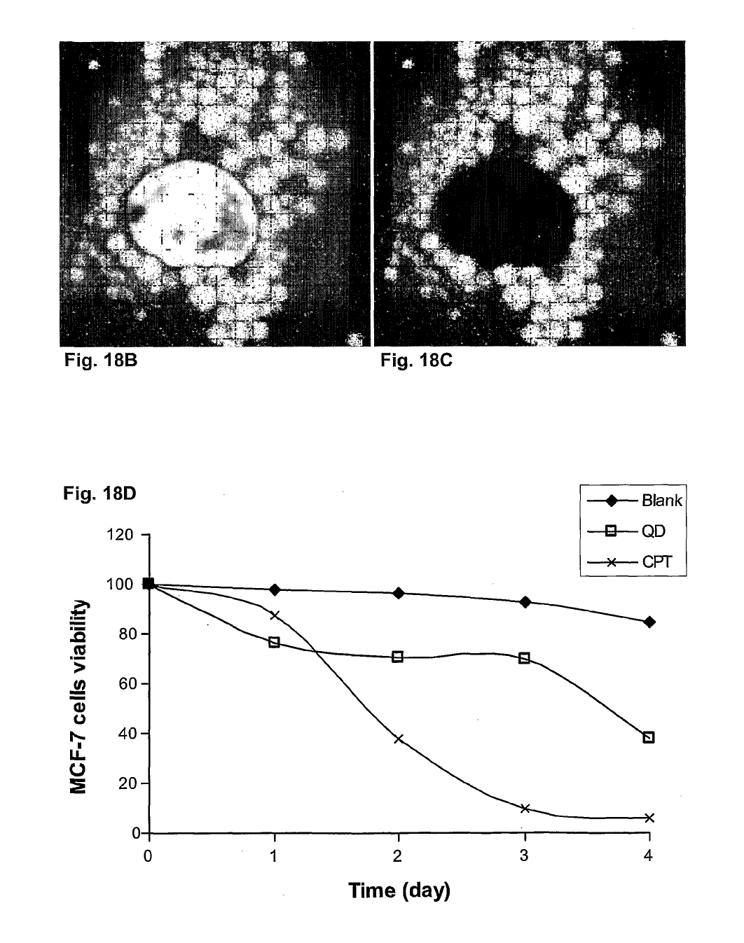

FIG. 18 shows optical property and biological application of QD and/or drug-incorporated microparticles. FIG. 18A is a confocal image of fluorescent drug incorporated microparticles. FIG. 18B is a confocal image of MCF-7 cell after incubation with blue drug-incorporated microparticles, which clearly shows cellular selectivity and uptake of microparticles made according to the present invention. The cell nuclei were stained red with PI. All channels are shown in FIG. 18B. FIG. 18C is the same confocal image as in FIG. 18B with only the blue channel shown, i.e. the fluorescent drug depicted, but not the cell nucleus. FIG. 18D depicts a graph that shows the efficacy of a potent drug delivery system shown by cell viability after treated up to 4 days with control placebo particles (blank), QD-loaded microparticles (QD), and drug-loaded microparticles (CPT).

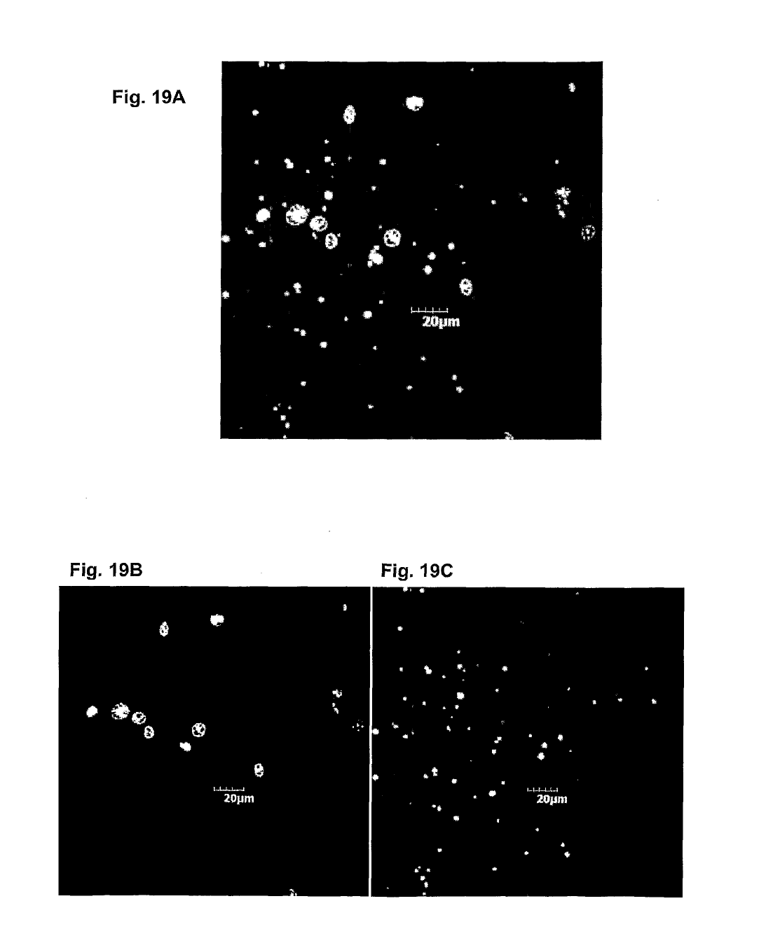

FIGS. 19 to 21 show selectivity in the uptake of drug-loaded microparticles by normal, healthy cells and by cancer cells. Results are similar to the selectivity of QD-loaded microparticles.

FIG. 19A is a confocal image of CCD-112CoN cells (healthy non-cancerous cells) after treatment with fluorescent drug-loaded microparticles. Cellular nuclei were stained red with PI. This is an overlaid image of the corresponding blue and red channel images. FIG. 19B is the confocal image of FIG. 19A with only the red channel shown, i.e. the cell nucleus depicted, but not the drug-loaded microparticles. FIG. 19C is the confocal image of FIG. 19A with only the blue channel shown, i.e. the fluorescent drug in the microparticles depicted, but not the cell nucleus.

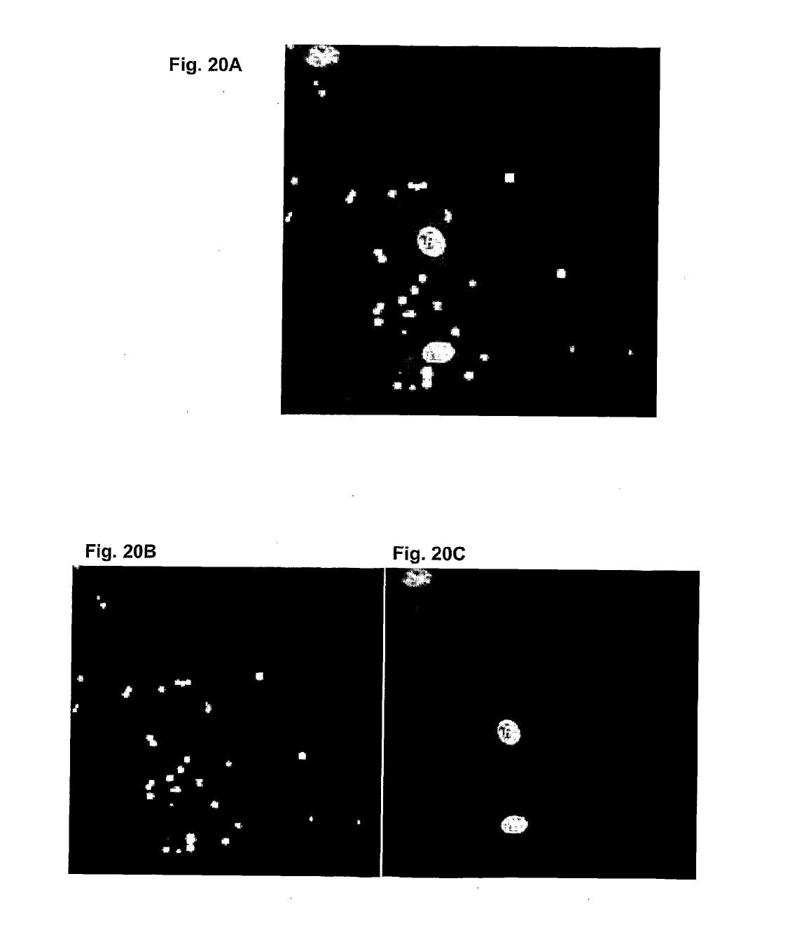

FIG. 20A is a confocal image of NIH3T3 cells (healthy non-cancerous cells) after treatment with fluorescent drug-loaded microparticles. Cellular nuclei were stained red with PI. This is an overlaid image of the corresponding blue and red channel images. FIG. 20B is the confocal image of FIG. 20A with only the blue channel shown, i.e. the fluorescent drug in the microparticles depicted, but not the cell nucleus. FIG. 20C is the confocal image of FIG. 20A with only the red channel shown, i.e. the cell nucleus depicted, but not the drug-loaded microparticles.

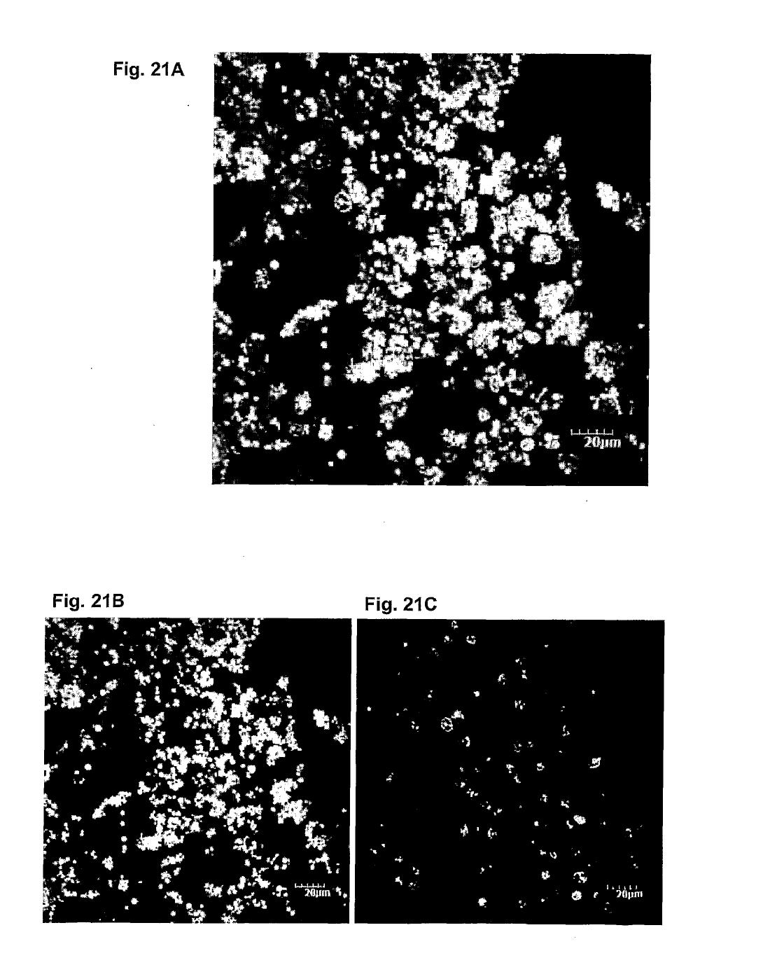

FIG. 21A is a confocal image of C6 cells (cancer cells) after treatment with fluorescent drug-loaded microparticles. Cellular nuclei were stained red with PI. This is an overlaid image of the corresponding blue and red channel images. FIG. 21B is the confocal image of FIG. 21A with only the blue channel shown, i.e. the fluorescent drug in the microparticles depicted, but not the cell nucleus. FIG. 21C is the confocal image of FIG. 21A with only the red channel shown, i.e. the cell nucleus depicted, but not the drug-loaded microparticles.

In order that the invention may be readily understood and put into practical effect, a particular embodiment will now be described by way of the following non-limiting example. It is understood that modification of detail may be made without departing from the scope of the invention.

### EXAMPLES

### Example 1

### Production of Quantum Dots (QD)/Nanoparticles-Incorporated Microparticles

These microparticles are prepared by a modified emulsification/solvent-evaporation method, and formulated by using a biocompatible amphiphilic copolymer as the matrix material. As a biocompatible polymer the FDA approved poly(DL-lactide-co-glycolide) (PLGA) is chosen for the present example (P2191, Sigma).

CdSe/ZnS quantum dots were obtained using published protocols, for example disclosed in Zhong, X H, et al., J. Am. Chem. Soc. (2003) 125, 8589-8594; Zhong, X H, et al., J. Am. Chem. Soc. (2003) 125, 13559; Peng, Z A, & Peng, X, J. Am. Chem. Soc. (2001) 123, 183; Yu, W W, Peng, X, Angew. Chem. Int. Ed. (2002) 41, 2368; Qu, L, & Peng, X, J. Am. Chem. Soc. (2002) 124, 2049). Briefly, in some experiments cadmium stearate (0.2044 g, 0.3 mmol), stearic acid (0.1707 g, 0.6 mmol), trioctylphosphine oxide (TOPO; 5.0 g) and octadecylamine (5.0 g) were added to a flask, and the mixture was heated to 310-330° C. under Argon flow until a clear solution formed. At this temperature, an excess amount of Se solution (0.1184 g, 1.5 mmol Se) in trioctylphosphine (4.0 g) was swiftly injected into the reaction flask, whereafter the temperature was set at 270-300° C. After 5-10 min, heating was stopped to stop the reaction and allow the flask to cool to room temperature. 3 mL of the as-prepared crude CdSe reaction mixture containing 0.1 mmol of CdSe was reheated to 290-320° C. At this temperature, ZnEt₂ solution (0.2 M) in TOP and Se solution (0.2 M) in TOP were added alternatively at time intervals of 20 s. After the addition, the reaction mixture was heated until no further PL peak shift (about 3-6 min). Heating was stopped to stop the reaction. The ratio of Zn/Cd in each targeted ZnxCd₁₋ₓSe nanocrystals can be achieved by the alteration of the amounts of Zn and Se precursors.

In other experiments, a mixture of CdO (0.0064 g, 0.05 mmol), ZnO (0.0081 g, 0.10 mmol), oleic acid (0.5 mL), and octadecene (4.0 mL) were heated to ˜80° C. and degassed under a vacuum of 10 Pa for 20 min. The reaction vessel was then filled with argon, and its temperature was increased to 310° C. After the CdO and ZnO precursors were dissolved completely to form a clear colorless solution, the temperature was lowered to 300° C. A solution of sulfur (1.0 mL, 0.0032 g, 0.10 mmol) in octadecene was swiftly injected into this hot solution within 1 s. In some experiments the reaction mixture was kept at 300° C. for the subsequent growth and annealing of the resulting nanocrystals. In other experiments the temperature was changed to 250° C. Aliquots of the sample were taken at different time intervals, and UV-vis and PL spectra were recorded for each aliquot. These sampling aliquots were quenched in cold chloroform (25° C.) to terminate growth of the particles immediately.

In a typical procedure in preparing nanoparticles (QD)-incorporated microparticles, 100 mg PLGA is dissolved in 4 ml dichloromethane (MeCl₂) (DR0440, Tedia) (acting as a non-polar solvent), and mixed together with 15 mg purified nanoparticles dispersed in 1 ml MeCl₂. A mixture of water and poly (vinyl alcohol) was used as the polar solvent. 30 ml aqueous solution of poly (vinyl alcohol) (PVA, 2% w/v) (P8136, Sigma) (acting as a polar solvent) were provided. The nanoparticles/PLGA solution (oil phase) was added into this aqueous solution under magnetic stirring at 400 rpm. The resultant solution was subjected to a homogenizer (T25, IKA) for emulsification at 6,000 rpm for 2 min at room temperature. After evaporating MeCl₂ from the oil-in-water emulsion, nanoparticles-incorporated microparticles were collected by centrifugation at 6,000 rpm for 10 min and further cleaned with deionized water for 3 times to remove excess PVA, nanoparticles, etc. When preparing drug- or nanoparticles/drug-incorporated microparticles, the drug was dissolved in oil phase for hydrophobic drug and aqueous phase for hydrophilic drug. Using this protocol nanoparticles were localized next to the surface in the obtained microparticles.

SEM images were taken and analyzed with the SmileView program (obtained from Jeol Ltd., Japan) to determine the average size and size distribution of the obtained particles. In the SmileView program, mean size is determined by measuring the size of 60 particles in the SEM image and the size distribution can be determined from standard deviation.

### Example 2

### Cell Uptake of PLGA Particles

Human colon fibroblast cells (CCD-112 CoN), rat fibroblast cells (NIH/3T3), human non-small lung cancer cells (CRL-5803), human breast cancer cells (MCF-7 cells), rat glioma cells (C6) were obtained from American Type Culture Collection (ATCC). Cells were maintained in DMEM medium supplemented with 10% FBS, 1.0 mM sodium pyruvate, 0.1 mM non-essential amino acids and 1% penicillin-streptomycin solution, and culture medium was replenished every other day. Cells were seeded at 2.0×10⁴ cells/cm² in Lab-Tek chambered cover glasses (Nunc) and cultured as a monolayer at 37° C. in a humidified atmosphere containing 5% CO₂. The cell uptake was started when the culture medium was replaced by particles solution (500 μg/mL in culture medium) and the monolayer was further incubated for 2 hr or 4 hr at 37° C. At the end of experiment, the cell monolayer was washed 3 times with fresh pre-warmed PBS buffer to eliminate excess nanoparticles which were not associated to the cells. Cells were then fixed with 70% ethanol. Nucleus staining was carried out using PI or DAPI to facilitate determine the location of the particles in the cells. The samples were then mounted in the fluorescent mounting medium (Dako). Confocal fluorescent microscopy was performed using an Olympus FV500 system supported with a 60× water-immersion objective. Images were captured in section of 1024×1024 pixels without zoom and with a 0.0-5.0 μm z-step, and processed by FV10-ASW 1.3 Viewer.

### Cell Viability

Cell viability was tested with human breast cancer cells (MCF-7 cells) applying the established colorimetric MTT assay. Cells were seeded at 2.3×10⁴ cells/cm² in the 96-well plates for quantitative cytotoxicity experiments and cultured as a monolayer. Cells were incubated with the culture medium containing drug-encapsulated microparticles for 1 to 4 days at 37° C. in a humidified atmosphere containing 5% CO₂. The viability or efficacy of the microparticles on cells was evaluated by measuring the optical density at 570 nm with a reference at 630 nm, using a microplate reader (Tecan). The optical density is directly proportional to the number of viable cells.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A microparticle comprising a biocompatible polymer, a nanoparticle, and an anti-cancer agent or a pharmaceutically acceptable salt, ester, prodrug or hydrate thereof, wherein the microparticle has a width of at least about 1 micron.

2. The microparticle of claim 1, wherein the nanoparticle is a quantum dot.

3. The microparticle of claim 1 or 2, wherein the microparticle has a width of at least about 2 microns.

4. The microparticle of any one of claims 1-3, wherein the microparticle has a width of maximally about 5 microns.

5. The microparticle of any one of claims 1-4, wherein the biocompatible polymer is a biodegradable polymer.

6. The microparticle of claim 5, wherein the biodegradable polymer is one of a poly(alpha-hydroxy acid), a polyalkylcarbonate, polycaprolactone, polyhydroxybutyric acid, a polyanhydride and a polyorthoester and combinations thereof.

7. The microparticle of claim 6, wherein the poly(alpha-hydroxy acid) is one of a polylactide, a polyglycolide, a poly(lactide-co-glycolide), and combinations thereof.

8. The microparticle of any one of claims 1-7, comprising a plurality of nanoparticles.

9. The microparticle of claim 8, wherein the nanoparticles are comprised in the micro-particle in an amount of 0.1 to 50 wt % of the biocompatible polymer.

10. The microparticle of any one of claims 1-9, wherein the anti-cancer agent is comprised in the microparticle in an amount of 0.1 to 20 wt % of the biocompatible polymer.

11. The microparticle of any one of claims 8-10, wherein at least essentially all of the nanoparticles are arranged proximate the surface of the microparticle.

12. The microparticle of claim 11, wherein at least essentially all of the nanoparticles are arranged in a near-surface zone of the microparticle, the near-surface zone being defined by the distance of 0 to about 250 nm from the surface of the microparticle.

13. The microparticle of any one of claims 1-12 for use in at least one of cancer therapy, cancer diagnostics and cancer therapy monitoring.

14. A process of forming a microparticle, the microparticle having a width of at least about 1 micron, wherein the microparticle comprises a nanoparticle, the process comprising:
providing a polar solvent and providing a non-polar solvent, the polar solvent and the non-polar solvent being at least substantially immiscible with each other,
dissolving the biocompatible polymer in one of the polar solvent and the non-polar solvent,
dispersing the nanoparticle in one of the polar solvent and the non-polar solvent, combining the non-polar solvent and the polar solvent, thereby forming a two-phase mixture, and
agitating the two-phase mixture, thereby allowing the formation of a microparticle.

15. The process of claim 14, wherein the nanoparticle is a non-polar nanoparticle and the biocompatible polymer is an amphiphilic polymer, the process comprising:
dissolving the biocompatible polymer and dispersing the nanoparticle in a suitable non-polar solvent, thereby forming a non-polar phase,
adding to the non-polar phase a polar solvent, the polar solvent being at least substantially immiscible with the non-polar solvent, thereby forming a two-phase mixture, and
agitating the two-phase mixture, thereby allowing the formation of a microparticle.

16. The process of claim 14 or 15, wherein the non-polar solvent and the polar solvent are used in a ratio of non-polar solvent/polar solvent in the range from about 1:10 to about 1:2.

17. The process of any one of claims 14 to 16, wherein the microparticle further comprises an anti-cancer agent or a pharmaceutically acceptable salt, ester, prodrug or hydrate thereof,
wherein the anti-cancer agent or the pharmaceutically acceptable salt, ester, prodrug or hydrate thereof is dissolved in one of the non-polar solvent and the polar solvent.

18. A method of treating cancer, the method comprising administering to an organism suffering from cancer a microparticle, the microparticle comprising a biocompatible polymer and an anti-cancer agent or a pharmaceutically acceptable salt, ester, prodrug or hydrate thereof,
wherein the microparticle has a width of at least about 1 micron.

19. The method of claim 18, wherein the microparticle further comprises a nanoparticle.

20. The method of claim 18 or 19, wherein the microparticle is predominantly taken up by cancer cells when compared to healthy cells.

21. A method of selectively directing an anti-cancer agent, or a pharmaceutically acceptable salt, ester, prodrug or hydrate thereof, to one or more cancer cells within a plurality of healthy cells, the method comprising:
providing a cell population, the cell population comprising a plurality of healthy cells and one or more cancer cells,
contacting the cell population with a microparticle, the microparticle comprising a biocompatible polymer and an anti-cancer agent or a pharmaceutically acceptable salt, ester, prodrug or hydrate thereof,
wherein the microparticle has a width of at least about 1 micron.
